(19) United States
(12) Patent Application Publication
(10) Pub. No.: US 2026/0229340 A1
(43) Pub. Date: Aug. 6, 2026

(54) Promises-Tracking Device and Method Thereof for Enhancement of Behavior Control

(71) Applicant: Deborah L. Hill, Sunland, CA (US)

(72) Inventor: Deborah L. Hill, Sunland, CA (US)

(21) Appl. No.: 19/045,448

(22) Filed: Feb. 4, 2025

(51) Int. Cl. G16H 20/70; G16H 20/60

(52) U.S. Cl. CPC G16H 20/70; G16H 20/60

(57) ABSTRACT

A promises-tracking device and method thereof is provided, wherein the promises-tracking device is configured to allow a participant to generate a physical activation signal through an activator thereof when each of a plurality of viable promises is made by the participant and to form a promise chain of the plurality of viable promises in order and store in a memory module thereof. The promise chain is broken when an input of a broken promise is selected by the participant via one of an excused broken promise button and an unexcused broken promise button of an input device of the promises-tracking device is received and determined, wherein only an unexcused broken promise is broken has a negative effect on a promise score of the participant.

### CROSS REFERENCE OF RELATED APPLICATION

This application is a Continuation-In-Part application that claims the benefit of priority under 35 U.S.C. § 120 to a non-provisional application, application Ser. No. 13/986,690, filed May 24, 2013, which is a Continuation-In-Part application that claims the benefit of priority under 35 U.S.C. § 120 to a non-provisional application, application Ser. No. 10/222,321, filed Aug. 16, 2002, incorporated herewith by references in their entireties.

### BACKGROUND OF THE PRESENT INVENTION

### Field of Invention

The invention relates to a promises-tracking device and method thereof for enhancement of personal control to help people increase their levels of success at performing desired behaviors.

### Description of Related Arts

Mind-tools, such as mental pictures, memories, moods, senses, ideas, plans, rationalizations, chemicals, nerve impulses, etc., are “abilities” that the mind can use to stimulate people to act. However, it is incredibly difficult and unpleasant for people to simply use their mental wills and mind-tools to control behaviors and to resist temptations that they feel they shouldn't have.

To illustrate the problem of behavior control merely by mind, an example of an over-eater (an individual who eats too much due to a lack of personal control), Albert, illustrated. For example, fat Albert is sitting on his sofa in his living room, trying to resist having a pie available in his kitchen because a member of his family, who is not on a diet, brought it home, that's not allowed on his diet. Albert will have to try to resist his desire for the pie as his powerful mind stimulates him or her to have it by:
(i) flashing mental pictures of the pie into his consciousness;
(ii) giving him or her cravings, urges, ideas and plans for having it;
(iii) sending him or her memories of how rich and creamy it tastes, as well as memories of feeling of enjoyment and well-being he derives from eating such comfort foods;
(iv) making his mouth water when he thinks about eating it;
(v) making sure that he notices other family members enjoying it;
(vi) creating a mental and physical state of readiness for it, such as quickening his heart rate and enlarging his pupils when he sees it;
(vii) giving him or her rationalizations and excuses for making an “exception” to his diet;
(viii) proposing deals for making an exception to his diet, for example, proposing a deal that if he has the illegal sweet “today”, he will not eat any sweets “tomorrow”, where such deals are often not kept, since Albert's mind will work just as hard for the next temptation:
(ix) minimizing the negative consequences of having the pie;
(x) maximizing the positive aspects, the pleasures, of having the pie;
(xi) creating a sense of urgency to have the pie;
(xii) intensifying the stimulations for having some of that pie;
(xiii) sending his urges to take steps in the direction of his kitchen, and urges him to reach out to take some;
(xiv) altering other known and unknown bodily chemicals and nerve impulses to assist in increasing and obtaining the desire for him;
(xv) altering his mood so that he feels that he can't accept doing without it;
(xvi) creating situations and scenes that will help him or her the pie he desires;
(xvii) making him or her feel unbearably sad, deprived, or even angry if he doesn't get some; and etc.

The above example of Albert illustrates just how incredibly difficult and unpleasant it is for people (for example Albert and others) to resist temptations that they feel they shouldn't have. (Over-eaters should not be confused with people who have personal control over how much they eat.) Mind-tools refers to all “abilities” that the mind can use to stimulate people to act, such as mental pictures, memories, moods, senses, ideas, plans, rationalizations, chemicals, nerve impulses, and etc.

The reason the mind works for things we desire, even when we feel we shouldn't have them (whether it be excess food, alcohol, cigarettes, illegal drugs, playing hooky, and etc.) is well known. Put simply, the primitive mind is ruled by the “Pain and Pleasure Principle”, that is it seeks pleasure and to avoid pain. So, it has a natural tendency to work for whatever we find pleasurable. In the case of fat Albert as exampled above, food is high on his list of things that bring him or her pleasure. So, once a desire for an available food temptation is kindled in his mind, he must “fight” his primitive mind to try to resist it, which can make it very difficult and unpleasant for him or her to resist.

The primitive mind is so powerful that when it works for such desires, success rates for resisting them are often very unreliable and low. And even if Albert is able to resist many temptations, he can make up for most or all of the progress he has made on his diet in a few moments, hours, or days of weakness. This is the reason for the current runaway obesity epidemic in the United States and other developed countries.

How to resist inappropriate temptations for people like Albert as the above example so that they can stick to their diets, exercise and other plans to achieve their goals is the traditional question to all people. Conventional methods mainly rely on mental processes and willpower for control in personal situations or try to control personal behaviors which require willpower such as fighting the mind, changing the mind, suppressing the mind, or dealing with the damages caused by the mind.

However, none of these conventional mental methods can be relied on for control in personal situations or personal behaviors that require willpower (for example fat Albert sitting on his sofa, trying to resist the pie in his kitchen) because mental processes, including diets, support methods, barriers and avoidance systems and willpower, which attempt to “fight” the mind are designed to help the over-eater fight the stimulations that his primitive mind sends as follows:

### Diets

The problems of diets are that the restriction and deprivation of diets only increase the stimulations from the primitive mind that the individual will have to fight. Diets which restrict certain foods, such as low carbohydrate or low fat diets, are usually ineffective, since people often begin to experience extremely strong cravings for the food combinations that they prefer and, sooner or later, give into them due to their primitive minds. It is now widely acknowledged that diets don't work. Long-term success rates of diets, even 3% are rare, and these low success rates are well documented.

Decades ago, the Washington Post reported that only one individual out of every 200 who go on a diet will lose all the weight they set out to and keep it off for any reasonable length of time. That is a 99.5% long-term failure rate and things have only gotten worse. Most studies report that less than 10% of dieters can maintain even a 5% loss from initial body weight for 5 years. For example, of those who start at 250 pounds, less than 10% can maintain a loss of even 12.5 pounds for 5 years. Of those who start at 150 pounds, less than 10% can maintain a loss of even 7.5 pounds for 5 years. As a result, the prevalence of obesity in the United States is only increasing, up 33% in the past decade alone.

Diets are very stressful and difficult to follow. Stimulations from the primitive mind make diets so stressful and difficult to follow that over-eaters frequently break or quit them. Then people can regain all the weight they may have lost in a few hours, days, or weeks of unrestricted eating and may even put on more weight.

Diets tend to make people more obsessed with the foods and quantities they shouldn't have. This can lead to obsessive, compulsive eating disorders, binging and purging, which can have severe physical and psychological consequences. Very often, people gain all their weight back, plus “a little more” each time they diet and it becomes more difficult to start and succeed each time they try, due to these resulting eating disorders.

Dieting can be unhealthy. Doctors now discourage very low calorie medically supervised diets (as were frequently prescribed in the recent past). According to the Merck medical manual, studies have shown that “. . . people tend to regain weight after going off the diet.” There is evidence that these diets, and yo-yo dieting, may damage a person's gallbladder, metabolism, and etc.

People on low carbohydrate diets may increase their risks for cancer, heart disease and other health problems, since these diets encourage more animal proteins and fats, and less healthy fruits and vegetables. A single apple has all the carbohydrates that many low carbohydrate diets may allow in a day, and the dieter would rather spend his “daily allotment” of carbs on nutrient-poor bread, pasta, sweets, chips or beer, instead of a healthier choice.

### Support Methods

Support methods are intended to help people fight the stimulations that their minds send to tempt them off their plans. The over-eater can seek support from his doctor, a counselor, a software program, motivational meetings, a personal trainer, an online group, a “call buddy system”, and etc. Such programs work best when they stress the negative aspects of the undesirable habits, educate about preferable one and give motivation, such as U.S. Pat. No. 4,344,625, entitled “Game Encouraging Self-Improvement”.

Support method through meetings and appointments can be expensive and inconvenient. There may be little or no support between meetings (when support is needed most). Setbacks are so common that dealing with them is built into the program. People who hire expensive personal trainers for support must schedule and keep appointments and are often embarrassed to sweat and grunt in front of a perfectly toned trainer, which can cause them to quit.

Support method through computerized programs can provide support for all aspects of planning, starting and following a weight loss plan and giving motivation, tips and incentives (such as rewards and punishments), such as U.S. Pat. No. 5,697,790 entitled “Discipline System”. But it takes a very disciplined individual to enter all the data into a computer and keep up with it constantly where over-eaters often lack the self-discipline. And the token tips, rewards and punishments are no match for the powerful primitive mind when it is working for a desire. After each failure, it's difficult and unpleasant to log in.

“Buddy Systems” is another support method that offers support during moments of temptation. For example, when Albert is sitting on his sofa trying to fight off his desires to have that piece of pie, he can log on to a chatroom or call a “buddy” to try to talk him or her out of it. But when Albert is tempted by a food desire, he will have to fight his mind just to log on or call his “buddy”. Even if he succeeds at taking that step, the buddy may or may not be able to talk him or her out of it. In addition, it is a common occurrence that even if that works, Albert's powerful mind can break down his willpower a few minutes or hours afterwards.

### Barriers and Avoidance Systems

Methods through barriers and avoidance systems attempt to create some kind of physical barrier between the individual and temptations.

Keeping Temptations Out of One's Environment: Albert of the above example could try to keep tempting pie out of his house. Variations of these plans include avoiding certain aisles in grocery stores, locking foods up and keeping junk foods out of schools. But these methods are highly impractical and unreliable in today's modem society. Albert won't be very popular with his family if he asks them not to bring home any food that's not allowed in his diet. And he will still have to fight his mind to avoid tempting locations, or could be surprised by them in the lunch room or at a party or meeting. There is no “eating police” to stop him or her from going out for them himself when tempted!

Weight Loss Resorts: People can check in to so-called “fat farms”, such as resorts where only healthy, low-calorie portions are served for days or weeks. But such places don't usually change a person's eating habits and people can't stay in them forever. When they are released, they often return to their old habits and the weight returns. Anyone who has ever stayed in such a place knows that his or her mind is so powerful that it can almost always find a way to get them forbidden temptations—like a $50 smuggled-in brownie.

Bodily Restrictions: Stomach capacity reduction surgeries do temporarily stop the mind from sending stimulations to eat too much by actually making it painful and dangerous for the over-eater to do so. But these procedures are expensive and carry significant physical, and even psychological, risks. Weight can quickly be regained as soon as the patient realizes that he can eat just as many calories each day by eating smaller portions more often or fattening foods in a liquid or semi-liquid state, such as mocha latte shakes and soft creamy pasta. The stomach and esophageal walls also tend to stretch over time, canceling out the food capacity-limiting effect. Wiring teeth shut make it difficult to speak and people quickly learn that straws can deliver high calorie ice cream shakes right through them.

Limited Portions of Food: Modem versions of the barrier method include buying portioned meals or having them delivered to one's home to limit portions. But people often dislike the taste of these low-fat, low-calorie, high fiber foods or simply start craving their own favorite brands, recipes and quantities. There is no “eating police” who will burst onto the scene and prevent the over-eater, Albert, from eating more than his boxed portion. If he is not full or wants something else, he will have to fight his powerful mind to try to stop eating.

In view of above, all of the above methods ultimately depend on ordinary willpower. For example, when Albert is trying to resist the stimulations, his mind is sending him or her to have that piece of pie, that he will have to fight his mind, using unreliable and wavering willpower. Willpower can be strong when a person is motivated, but weak and unreliable when the person is tempted just when he needs it most. It is because the mind-tools give the primitive mind nearly limitless power, failure is common.

Willpower is a relatively easy obstacle for the powerful primitive mind to overcome to get people to give in to their desires. In fact, who knows a person better than his own mind? Albert's mind knows just the right rationalizations to use to get him or her to give in to that pie. It knows just the right moods to put him or her in; the right scenes to create; and the best plans and ideas to send into his consciousness to tempt him or her to have it. Even if he succeeds at resisting, it will be a very difficult, stressful and unpleasant experience for him.

On the other hand, other methods which attempt to “change” a person's mind endeavor to reduce or eliminate the urges sent by his mind to do what he feels he shouldn't do. These include education, behavior modification, analysis (of one's childhood or past), hypnosis and positive affirmation methods, positive reinforcement and feedback methods, rehearsal methods, and self-esteem training, each of which has unsolved problems as follows.

EDUCATION: Teaching people that fatty, sugary food is bad for them can reduce the urges to have it for some, but not usually for over-eaters. Over-eaters typically eat based more on what they currently want (instant gratification), rather than what's good for them. For example, just teaching fat Albert that certain foods are healthier does not mean that he will choose them. Albert will have to fight his mind to try to resist having the pie he wants, regardless of how much he knows he shouldn't have it. His knowledge will only increase his feelings of guilt and deprivation. Healthier foods are generally more time consuming to prepare. Low fat foods don't taste as good and aren't as satisfying as the fast, easy, high caloric foods that are constantly advertised to consumers. Companies that produce low fat foods are reporting that people have been turning away from their products and buying foods that taste better.

BEHAVIOR MODIFICATION: Behavior modification methods, such as U.S. Pat. No. 5,207,580, entitled “Tailored Health-Related Behavior Change and Adherence Aid System”, are provided to teach the over-eater to eat without distractions, eat more slowly, take small bites, put his fork down between each bite, and etc. The patented method of tailoring aid to an individual to change an undesirable health-related behavior of the individual to a selected desirable health-related behavior and to maintain the selected desirable health-related behavior, comprising steps of:
(a) obtaining answers from the individual to questions related to topics selected from the group consisting of:
(i) characteristics of said individual's undesirable health-related behavior;
(ii) factors motivating said individual to change the undesirable behavior; and
(iii) factors acting as barriers to changing the undesirable behavior;
(b) providing a plurality of instruction statements, the plurality comprising preparation phase statements, change phase statements and maintenance phase statements, each of the statements being tailored to possible answers to the questions;
(c) determining a change date for changing the individual's undesirable behavior;
(d) providing one or more dated pages comprising a space for said change date and having dated spaces for preparation dates prior to the change date and for maintenance dates after the change date;
(e) marking the change date in the space for the change date and placing one or more change phase statements in the space for the change date;
(f) placing one or more preparation phase statements in spaces for selected preparation dates which occur prior to the change date;
(g) placing one or more maintenance phase statements in spaces for selected maintenance dates which occur after the change date; and
(h) providing the dated pages to the individual before the preparation dates.

The steps of U.S. Pat. No. 5,207,580 patent is not directed to an abstract idea since specific physical steps are executed by the individual so as to cover performance of the limitations of the mind and the management of personal behavior. However, the method will not guarantee that the over-eater will do it. For example, fat Albert's primitive mind will fight such restrictions. Setbacks are common; and significant long-term improvements are rare, especially for over-eaters.

ANALYSIS: Blaming previous experiences for negative behaviors is controversial. For example, fat Albert may have had a traumatic experience when he was young, but psychologists disagree as to whether or not this could have caused his eating problem. Even if a lack of personal control can be traced to a past experience, this does little to correct the problem. Years of therapy aimed at correcting the problem are often plagued by setbacks. However, there is little doubt that a lack of personal control can cause physical and psychological problems. Albert's inability to control how much he eats will almost certainly lead to physical ailments, anxiety, low self-esteem, depression and even obsessive-compulsive tendencies. The real problem is often a lack of reliable personal control.

HYPNOSIS AND POSITIVE AFFIRMATION SYSTEMS: An over-eater, for example fat Albert, can be given a hypnotic suggestion to have an apple for dessert (instead of chocolate cake). Or, he may repeat such positive phrases (or affirmations) to himself. However, these methods have been shown to be highly ineffective ways of improving behavior for over-eaters, in both the short term and long term. The over-eater's preference for chocolate cake is often much stronger than a hypnotic suggestion. When these methods work it is often because of the placebo effect, that is the individual has been told by an “expert” that they will work, and the effect does not usually last.

POSITIVE REINFORCEMENT AND FEEDBACK METHODS: U.S. Pat. No. 5,954,510, entitled “Interactive Goal-Achievement System and Method, discloses a positive reinforcement and feedback method, which is a computer implemented method for assisting an individual to set and achieve goals, comprising the steps of:
(a) periodically collecting information about goals from an individual, including an individual's estimate of their likelihood of achieving the goals and progress towards said goals, by entering the information into an input device connected to a computer system;
(b) computing metrics with the computer system from the information, the metrics corresponding to the individual's progress towards reaching the goals; and
(c) providing feedback information to the individual regarding the individual's progress towards reaching the goals and the individual's estimate of their likelihood of achieving said goals, by communicating the feedback information from the computer system to an output device interfaced thereto.

A computer is implemented in the U.S. Pat. No. 5,954,510 patent to execute the information collection step (a), the computation step (b) and the information feedback step (c) from the computer for the individual to assist people in achieving and learning to achieve self-determined, measurable goals. It is not an abstract idea since specific physical steps are executed to cover performance of the limitations of the mind and the management of personal behavior. However, a low success rates persist despite such supportive positive reinforcement (or feedback) systems, such as the U.S. Pat. No. 5,954,510, and negative reinforcement (or feedback) systems, such as the pain-induced electrical punishment system applied to the skin while the patient engages in undesirable behaviors and is told their negative qualities, “The Schick Method.”

Some of these methods utilize equipment that is similar to pilot training simulators to help people know and practice what to do in specific situations. For example, fat Albert can watch a simulated scene and use a keyboard, a toggle switch, or be wired so that he can react to the scene. For instance, the screen may show him or her walking into a simulated break room and—uh oh—he is surprised by the enemy: an open box of donuts! He practices doing “the right thing”—grabbing a celery stick and walking away—and then records how much better he feels about doing so. This is repeated in an effort to strengthen nerve synapses for more desirable behaviors.

In this way, a simulator can help the over-eater know what to do at a time of temptation. But this cannot reliably eliminate the mind battles that he must fight in the real battlefields of temptation or guarantee that he will succeed. What fat Albert will do depends on whether or not he can win the fight against his infinitely powerful mind. When he really wants a food temptation, his primitive mind will usually succeed at getting it for him.

These methods are similar to all systems that are designed to help people rehearse doing the “right thing”, such as hypnosis, positive affirmations, counseling, journaling, meetings, and etc. But the primitive mind can easily override even strong nerve impulses when working for desires. Such is the case when fat Albert wants pizza on a cold night and has to go out for it. His mind can override his strong impulses to stay warm, dry and comfortable and can help him or her dress and brave the weather.

SELF-ESTEEM TRAINING: Behavioral scientists noticed that people who feel good about themselves have higher levels of personal control. So, they worked on increasing the self-esteem of over-eaters, theorizing that this would give them greater personal control. But this yielded disappointing results. It's just plain hard for an over-eater, like Albert, to feel good about himself when he keeps losing control. However, it is well known that increasing personal control can increase self-esteem dramatically.

Methods which attempt to “suppress” the mind include those which try to suppress hunger or desires, such as hunger suppressants and desire suppressants.

HUNGER SUPPRESSANTS: According to the Merck Medical Manual, regarding hunger suppressants such as amphetamines, “There is a danger in using these drugs because they simultaneously overexcite the central nervous system, making the person nervous and elevating blood pressure. Also, a person soon adapts to the drug, so that weight reduction is usually no more than 5 to 10 percent.”

DESIRE SUPPRESSANTS: Some conventional methods are designed to reduce desires for temptations, such as dulling the taste buds or blocking smell, so that food doesn't taste as good. These methods never became very popular because they prevented people from enjoying eating, which is especially important to the over-eater.

Other conventional methods attempt to “deal with the damages” caused by the mind's stimulations don't attempt to stop the negative behaviors; they simply try to deal with the aftermath but the resulting weight gain obesity, low self-esteem, health problems, and etc. These conventional methods include exercise, fat and sugar blockers, prescription drugs, girdles and “slimwear”, and self-acceptance training, each of which has problems as follows.

EXERCISE: Excessive exercise is very stressful for the human body. For example, after failing on many stressful diets, Albert could decide to just give up on dieting and run 10 miles a day to “bum off” all the calories he eats. He could wear out or injure his joints. Also, exercise is simply not very effective. Despite what exercise promoters lead people to believe, it's a medical fact that it takes 17 days of doing one hour of “active exercise” (like brisk walking, calisthenics or using a recumbent bike) to lose a single true pound of fat. That is why advertisements for exercise methods almost always refer to them as a “plan” which includes a reduced calorie diet. Unfortunately, it only takes one day, or a few hours, of eating excessively to regain a pound lost by weeks of exercise. So exercise is not a solution for those who continue to over-eat.

FAT AND SUGAR BLOCKERS: Fat and sugar blockers often have unpleasant side effects (such as bloating and “leaking anus”), and they often result in compensating, as happened with the advent of sugar substitutes. For example, fat Albert could rationalize that he is “saving” calories and eat more. These products usually only block small amounts of fats and sugars, so the net result is often weight gain and the dissatisfied participant stops using them.

PRESCRIPTION DRUGS: An individual, for example fat Albert, could ask his doctor to give him or her something to speed up his body's metabolism so that it will “bum off” the excessive calories he eats. But, according to Merck Manual, “Generally, such a drug reduces weight by about 10 percent within 6 months and maintains the loss as long as the drug is continued. When it's discontinued, the weight is promptly regained.” These drugs also carry substantial side-effect risks, which is why their use must be prescribed and closely monitored by a physician.

Many doctors will not give Thyroid, the hormone that increases metabolism, because so much is required that it gives extreme side effects. Additionally, the body will respond by lowering the thyroid output even further, canceling out its effect. Even if it were easy to significantly increase metabolism, there would be other negative effects, such as accelerated aging and increased risks for disease. Accelerated cell replication would result in accelerated replication mistakes, resulting in faster aging and diseases like cancer.

GIRDLES AND “SLIMWEAR”: Girdles and slimwear can't really hide the ugly truth or reduce the health hazards of obesity, and don't really solve the problem.

SELF-ACCEPTANCE TRAINING: The physical and mental stresses of dieting can be so severe that the dieter may conclude that it's better to just learn to accept himself as is. Indeed, dieting can actually increase the stimulations from the primitive mind, resulting in eating disorders that can make the over-eater obsessed with foods. But it's difficult for over-eaters to accept their unhealthy, unattractive bodies and accept themselves as failures. This is not the solution they prefer.

In view of above, none of the above conventional methods can be relied on to help people resist available temptations in personal situations. Although all methods work some of the time for some people, none work reliably or give lasting results for most people—otherwise we wouldn't have an obesity epidemic. Despite the vast body of information and technology that modem society has to offer, it is still very difficult and unpleasant for people like fat Albert to resist excessive food desires, because the primitive mind is so powerful and relentless, and their success rates for resisting are still extremely low and unreliable.

Failure is just as prevalent when it comes to following other kinds of personal plans. The majority of people who join gyms or buy exercise equipment don't use them for more than a few days or weeks, because their primitive minds fight their plans! People also have to fight their minds to follow plans to eliminate drug and alcohol abuse, to stop smoking, etc., resulting in the same low and unreliable success rates. Very few people can stick to their self-improvement plans long enough to achieve the results they want. An effective method is desperately needed.

### SUMMARY OF THE PRESENT INVENTION

The invention is advantageous in that it provides a promises-tracking device and a method thereof for enhancement of behavior control, which is a program operated electronic device configured to form a promise chain and a promise score for tracking promises of a participant that significantly helps the participant to resist temptations.

Another advantage of the invention is to solve the problems of the conventional methods mentioned in the background of the invention by means of a promises-tracking device for enhancement of behavior control, which is a program operated electronic device configured to form a promise chain and a promise score for tracking promises of a participant, so that the participant doesn't have to fight his or her mind to resist temptations.

Another advantage of the invention is to provide a promises-tracking device and a method thereof for activating the natural Mental Shielding ability of the people's minds “at will” so as to help helping them to create a new reliable superego influence.

Another advantage of the invention is to provide a promises-tracking method to enable people establishing their chain of promises and make a strong commitment to keeping the promises they add to the promise chain thereof.

Another advantage of the invention is to provide a promises-tracking device and a method thereof for enhancement of behavior control, which allow people to make a very long commitment to keeping the promises they add to their promise chains, so that they can reliably achieve the sureness required to program their minds to keep them.

Another advantage of the invention is to provide a promises-tracking device for enhancement of behavior control, which is a program operated electronic device configured to form a promise chain and a promise score for tracking promises of a participant, through executing true, immediate and undeniable steps by the participant while, optionally, being certified by a panel of psychologists and medical doctors who participated in a clinical test, which is different from the conventional nebulous, unreliable methods that claim to be able to program the mind, such as self-hypnosis and positive affirmations.

Another advantage of the invention is to provide a promises-tracking device and a method thereof for enhancement of behavior control, which is a program operated electronic device configured to form a promise chain and a promise score for tracking promises of a participant, that allow the participant to easily, comfortably and reliably withstand temptations that would otherwise be very difficult and unpleasant for the participant to resist, so as to give much higher success rates for execution of the promises-tracking method for enhancement behavior with the promises-tracking device of the invention.

Another advantage of the invention is to provide a promises-tracking device and a method thereof for enhancement of behavior control, which is a program operated electronic device configured to form a promise chain and a promise score for tracking promises of a participant, that significantly overcomes the major problems, including the lack of reliable personal control, the discomfort while resisting temptations and the success rates for sticking to the behavior plans, of the conventional methods mentioned in the above background of the invention.

Another advantage of the invention is to provide a promises-tracking device and a method thereof for enhancement of behavior control, which is a program operated electronic device configured to form a promise chain and a promise score for tracking promises of a participant, that enable the participant to activate the same programmed state of mind that the mind naturally activates to help the participant to resist temptations easily, comfortably and with a very high degree of reliability.

Another advantage of the invention is to provide a promises-tracking device and method thereof which also provides personal analysis, teaching, training, guidance, support, motivation and new applications for use of the Shield Method.

Another advantage of the invention is to provide a promises-tracking device and a method thereof for enhancement of behavior control, which overcomes major problems of the prior art by providing a means for self-control that is capable of withstanding strong urges and desires to do other than a person's specifically planned behaviors and achieving a high degree of success in keeping them.

Another advantage of the invention is to provide a promises-tracking device and a method thereof for enhancement of behavior control, which allows an individual does not have to be deprived of the foods he or she loves—he or she can enjoy less and stay in control.

Another advantage of the invention is to provide a promises-tracking device and a method thereof for enhancement of behavior control, which can be used by people to control personal behaviors, including but not limited to stop smoking, control drug, control weight, and alcohol consumption, for almost any other personal control applications as well, with or without modifications. follow exercise programs, and etc.

Another advantage of the invention is to provide a promises-tracking device and a method thereof for enhancement of behavior control, which is configured to help people form, commit to and effectively use a chain (or succession) of behavior promises in such a way that they can activate the Mental Shielding ability of their minds for keeping the promises they add to their promise chain with a high degree of reliability. When this mental shield state of mind is activated, it can effectively program the participant's mind to follow the behavior the participant has promised, altering activities of his or her mind and body to help them follow the behavior plan with a very high degree of reliability and success.

Additional advantages and features of the invention will become apparent from the description which follows, and may be realized by means of the instrumentalities and combinations particularly point out in the appended claims.

According to the present invention, the foregoing and other objects and advantages are attained by a promises-tracking device which is a program operated electronic device for forming a promise chain of a plurality of viable promises of a participant and a promise score for tracking the plurality of viable promises of the participant, wherein the promises-tracking device comprises:
at least one processor;
an input device comprising an excused broken promise button, an unexcused broken promise button, a time setter configured for setting promise times of the plurality of viable promises, an activator configured for the participant to generate a physical activation signal when each of the plurality of viable promises is made by the participant, wherein the excused broken promise button and the unexcused broken promise button are configured to be selectively inputted for an input of a broken promise when one of the plurality of viable promises is broken;
at least one memory module stored program to instruct the at least one processor to receive a plurality of viable promises inputted by the participant through the input device in a chainlike manner and store the plurality of viable promises to form the promise chain on the at least one memory module; and
a screen configured to display the physical activation signal generated through the activator of the input device to notify while the viable promises is sealed in the promise chain, the setting promise times, in response to the plurality of viable promises of the promise chain in a numbering order in the memory module, being set via the time setter, a notification when the promise times are up, and a level of sureness of the participant graphically for the participant to view and periodically for monitoring the level of the sureness of the participant, wherein when the unexcused broken promise button is selectively inputted for the input of the broken promise while one of the plurality of viable promises is broken, the promise chain is then broken to have a negative effect on the promise score of the participant, which is determined and generated regarding the promise chain and displayed via the screen, wherein a level of sureness.

According to the preferred embodiment, the at least one memory module further instructs the at least one processor to determine whether the one of the pluralities of viable promises being broken is the excused broken promise or the unexcused broken promise by the at least one processor, wherein only the unexcused broken promise has a negative effect on the promise score.

According to the preferred embodiment, the promises-tracking device further comprises a selector which is configured to enable the participant to have a selection of a learning and training phase with one or more lessons for techniques of effective promise stored in the memory module according to a personal control score of the participant, wherein a baseline documentation is stored in the memory module and displayed via the screen, wherein the baseline documentation includes a plurality of personal, health and habitual questions for the participant to answer in order to obtain a personal control score for evaluating what kind of lesson and assignment in the learning and training phase to be displayed to the participant, wherein lesson material and suggestions are selected to the participant according to the personal control score through the selector, so as to help the participant to restore the sureness of the participant when said level of sureness drops.

According to the preferred embodiment, the promise score contains information selected from the group consisting of total promises added to chain, total broken promise, and percentage of successful promise.

In accordance with another aspect of the invention, the present invention further provides a promises-tracking method for forming a promise chain and a promise score for tracking a plurality of viable promises from a participant, executed by a program operated electronic device which comprises a screen, an activator, at least one memory module, and an input device including a time setter, an excused broken promise button and an unexcused broken promise button, wherein the program operated electronic device is a non-transitory computer readable medium containing computer instructions stores in the at least one memory module for causing the at least one processor to perform the promises-tracking method comprising steps of:
(a) recording the plurality of viable promises made by the participant in a chainlike manner through the input device of the program operated electronic device to form the promise chain and storing the plurality of viable promises of the promise chain on the memory module of the program operated electronic device;
(b) generating a physical activation signal through the activator of the program operated electronic device to notify while the viable promises is sealed in the promise chain;
(c) in response to setting promise times of the plurality of viable promises by the participant via the time setter of the input device of the program operated electronic device, numbering the viable promises in an order in the memory module;
(d) selectively notifying the participant via the program operated electronic device when the promise times are up;
(e) determining and generating a promise score of the participant regarding the promise chain, which is an incentive to motivate the participant to keep the promise score as high as possible, wherein when one of the viable promises is broken, the promise chain is then broken;
(f) determining whether one of the pluralities of viable promises being broken is an excused broken promise or an unexcused broken promise when one of the excused broken promise buttons and the unexcused broken promise button is selected to input by the participant, wherein only the unexcused broken promise is broken has a negative effect on the promise score; and
(g) providing a level of sureness of the participant on the screen, wherein the level of sureness of the participant is indicated graphically on the screen of the program operated electronic device for the participant to view and displayed periodically on the screen of the program operated electronic device for monitoring the level of sureness of the participant.

According to the preferred embodiment, the step (e) further comprises steps of:
(e1) receiving an input of a broken promise selectively by the participant via one of the excused broken promise buttons and the unexcused broken promise button of the input device of the program operated electronic device, and
(e2) displaying the promise score on the screen of the program operated electronic device.

According to the preferred embodiment, the step (g) further comprises a step of monitoring the level of sureness of the participant for ensuring the participant is able to keep the viable promises, wherein when the level of sureness drops, lesson material and suggestions are provided to help restore the sureness of the participant.

According to the preferred embodiment, the step (g) further comprises a step of monitoring the level of sureness of the participant for ensuring the participant is able to keep the viable promises, wherein when the level of sureness drops, lesson material and suggestions are provided to help restore the sureness of the participant.

According to the preferred embodiment, in the step (e), the promise score contains information selected from the group consisting of total promises added the promise chain, total broken promise and a percentage of successful promise.

According to the preferred embodiment, the promises-tracking method further comprises a step of providing a learning and training phase by the program operated electronic device to the participant, wherein in the learning and training phase, one or more lessons are given to the participant regarding effective promise techniques, and one or more assignments for the participant to complete regarding the promise techniques, wherein an assignment score of the participant is determined regarding a completeness of the assignment.

According to the preferred embodiment, in responsive to a selection of the participant through a selector provided on the input device of the program operated electronic device, the program operated electronic device provides at least a learning and training phase with one or more lessons for techniques of effective promise stored in the memory module to the participant according to a personal control score of the participant.

According to the preferred embodiment, a baseline documentation phase is provided and stored in the memory module, wherein a plurality of personal, health and habitual questions are given for the participant to answer in order to obtain a personal control score for evaluating what kind of lesson and assignment in a learning and training phase to be given to the participant, wherein lesson material and suggestions are selected to the participant according to the personal control score through the selector, wherein the lesson material and suggestions are provided to the participant via the screen of the program operated electronic device to help the participant to restore the sureness of the participant when the level of sureness drops.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a schematic view of a smart device programmed to be a promises-tracking device for executing a promises-tracking method according to a preferred embodiment of the present invention.

FIG. 2 is a schematic view of a tablet computer programmed to be a promises-tracking device for executing the promises-tracking method according to the above preferred embodiment of the present invention.

FIG. 3 is a block diagram of the promises-tracking device according to the above preferred embodiment of the present invention.

FIG. 4 is a block diagram illustrating the promises-tracking method according to the above preferred embodiment of the present invention.

FIG. 5 is a block diagram illustrating a locking mechanism of the promises-tracking method according to the above preferred embodiment of the present invention.

FIG. 6 is a block diagram illustrating the baseline documentation phase and the learning and training phase of the promises-tracking method according to the above preferred embodiment of the present invention.

FIG. 7 is a perspective view of a ring type activator, worn on a finger of a participant, of the promises-tracking device according to the above preferred embodiment of the present invention.

FIG. 8 is a block diagram illustrating a plurality of phases and steps of the promises-tracking method for the participant according to the above preferred embodiment of the present invention.

FIG. 9 is a block diagram of an alternative mode of the promises-tracking device according to the preferred embodiment of the present invention.

FIG. 10 is a schematic diagram illustrating a personal control scores display according to the preferred embodiment of the present invention.

FIG. 11 is a schematic diagram illustrating a test scores display according to the preferred embodiment of the present invention.

FIG. 12 is a schematic diagram illustrating an assignment scores display according to the preferred embodiment of the present invention.

FIG. 13 is a schematic diagram illustrating a promise scores display according to the preferred embodiment of the present invention.

FIG. 14 is a schematic diagram illustrating a promise scores display according to the preferred embodiment of the present invention.

FIG. 15 is a schematic view illustrating an exemplary level and category for the promises-tracking device and the method thereof according to the preferred embodiment of the present invention.

FIG. 16 is a schematic view illustrating the excused broken promise button and the unexcused broken promise button of an exemplary screen display for the promise-tracking device according to the preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

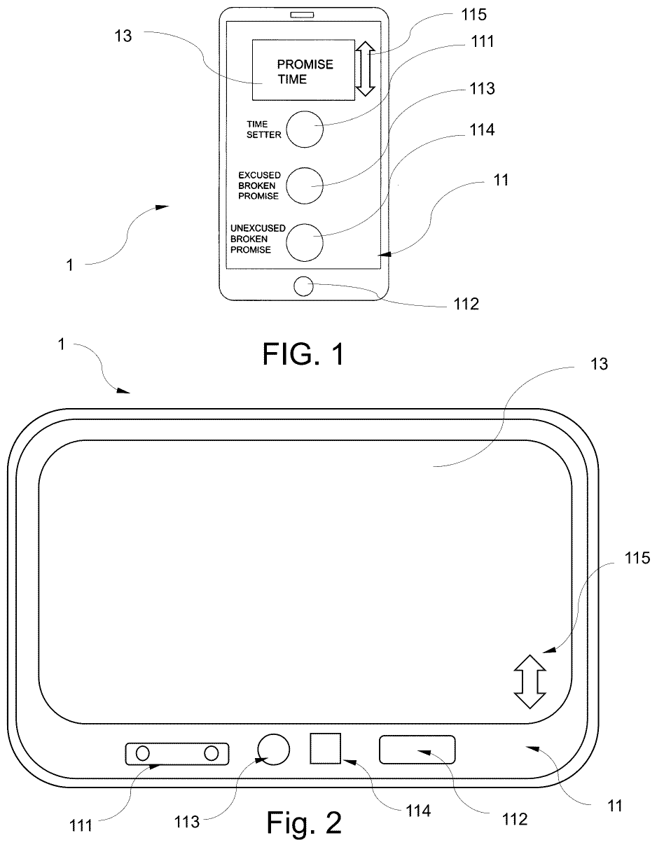

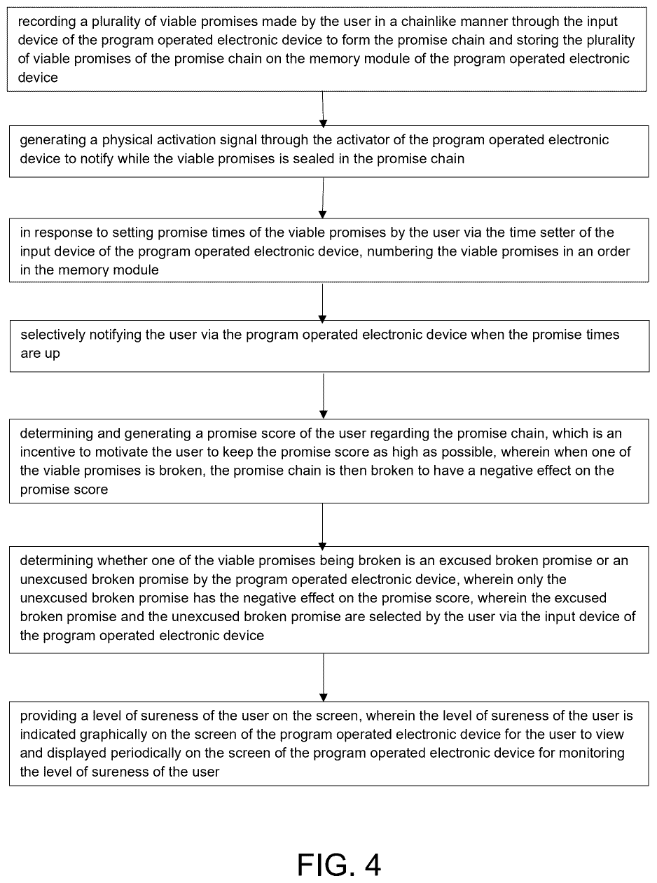

Referring to FIG. 1 to FIG. 4 of the drawings, a promises-tracking device and a method thereof for enhancement of behavior according to a preferred embodiment of the present invention are illustrated, wherein the promises-tracking device is a program operated electronic device configured to form a promise chain and a promise score for tracking promises of a participant.

In order to better illustrate the promises-tracking device and the method thereof of the present invention, the mechanism of how the mind being naturally used to control behavior of an individual is described as follows.

For example, an over-eater individual, fat Albert, is in one of those “big box” stores, like Costco, approaching a table where savory crackers topped with a delicious dip are being given out as samples. In this situation, since he is sure that he can only take one sample, his mind will be programmed to help him or her resist having more. Then, even an over-eater like Albert will be able to stay in control. It will be relatively easy for him or her to have just one sample, and he will be able to resist taking more. Even those who would go back for another sample, or two, will not start eating the food uncontrollably, like they might do at home. We don't ever see anyone lose control and be unable to stop eating at a sample table.

Note that because Albert's mind is programmed to help him or her resist taking more than the socially acceptable amount in this situation, he will not have to fight his mind to resist taking more. In fact, his mind will be “switched” to helping him or her resist more by using its mind-tools to help him or her accept that he can only have the limited amount and keeping him or her as comfortable as possible with just that much. One of the bonuses of this is that his mind will help him or her really concentrate on and enjoy the flavors and textures of the small sample benefit that sample-givers count on. His mind will help him or her easily and comfortably stay in control, while giving a remarkably high success rate for resisting more. The question is how to trigger and control such affirmative behavior of resisting temptations and feelings of sureness by the people themself.

Feeling sure that he could only have one sample is what activated the programmed mental state that kept the over-eater comfortable with the small amount, since “feelings of sureness” program the mind. But notice that having a sample at a promotion table in a market, like Costco, as mentioned above was a public (social) situation. There has never been a practical or reliable way for people to create this sureness, which can activate this natural ability of the mind, in personal situations that require willpower.

Although there are ways to create this kind of sureness for personal control, they are not practical or reliable. For example, fat Albert buys some of those crackers and dip from the big box store. When he gets them home, he could pour them into a large bowl and then pour molasses (motor oil) all over them and stir it all up, being careful to coat every cracker and completely saturate the dip with the oil. Then, this would certainly create the “sure” state of mind that he wouldn't eat them, which would program his mind to make it easy for him or her to resist them.

However, as mentioned above, this method is not practical or reliable. Albert would have to use willpower to pour molasses over the crackers and dip (he would have to fight his mind to do it), so there would be no advantage. And even if he were to succeed at saturating them with molasses, there would be plenty of other food temptations in his cupboards—or nearby 24 hour convenience stores—to pig out on. Also, he would be wasting food for why buy food to waste it. Therefore, there has never been a practical or reliable way to accomplish this kind of personal sureness to program the mind before the present invention.

According to the preferred embodiment of the present invention, examples of method to enable participants to enhance their personal behavior controls for “sureness” to program the mind of an individual which can make unpleasant behaviors that the primitive mind would fight much easier and more comfortable to accomplish, while giving very high success rates, usually over 99%, are illustrated.

It is worth mentioning examples of situation that people's minds will be programmed with sureness to help them to accomplish jobs as comfortably as possible due to social, safety, responsibility, religious and other influences as follows.

Firstly, since drivers feel sure that they must keep their eyes on the road and their hands on the wheel while driving, their minds are programmed to help them doing so rather than fighting this behavior. This enables them to cruise down freeways at high speeds for hours at a time, within a few feet of large SUV's and 18 wheeler trucks, as comfortably as possible, without making a serious driving mistake—over 99% of the time, comparing this to dieters who typically have eating accidents and diet crashes daily.

Secondly, because most mothers feel sure that they must change their babies diapers promptly, to protect them from discomfort, diaper rashes and unsanitary messes, their minds are programmed to help them accept that they must do this unpleasant task and help them accomplish the job, as comfortably as possible.

Thirdly, it's not easy to feed, house and clothe a child 365 days a year but, because parents feel sure that they must do it, due to social, religious and other influences, their minds are programmed to help them to accomplish. This enables them to take care of their children every single day, as comfortably as possible, and even, often, to find joy in doing so.

Fourthly, because soldiers feel sure that they will engage the enemy when ordered to, their minds are programmed to help them follow this very difficult behavior, as comfortably as possible, enabling them to accomplish great acts of bravery in combat situations.

If a driver feels sure that he is about to go through his front windshield in an accident, his mind will naturally be programmed to take every conscious and instinctual action it can to help him or her survive, as comfortably as possible. This may include blacking out the memory from his conscious mind to help him or her cope. Please note that the programmed mind can't always make people completely comfortable while doing their unpleasant things, but it will work to keep them as comfortable as possible.

According to scientific description, this programmed mental state is described in the field of psychology in terms of the “id”, the “ego” and the “superego”, the three divisions of the mind or psyche.

The “id” is the primitive mind (instinctual drives). The “superego” represents the moral standards and ethics that you believe in. It rewards you with good feelings about yourself when you meet these standards, and makes you feel bad when you don't. The “ego” mediates between the above two divisions, “id” and “superego” of the mind.

To clarify, taking example of an over-eater, like fat Albert of the above example, is approaching the sample table at Costco. Although he would prefer to eat samples without restriction (a primitive “id” desire), he feels sure that he can't in this situation (because of a strong social “superego” influence), so his “ego” programs his mind to forego (and protect him or her from) the “id” desire. This makes it easy and comfortable for him or her to resist having more. Only severely mentally impaired people can't do this.

According to the present invention, terminologies as used to describe inventive concepts, including “Mental Shield”, “Shield”, “Absolutely Sure”, “Shielded”, “Shielder”, “Shielding”, and “Shield Method”.

According to the preferred embodiment of the present invention, a sure-programmed state of mind as described above is called “Mental Shield,” since it protects (Shields) behaviors; and protects (Shields) people from feeling uncomfortable without desires. Specifically, a Mental Shield is a programmed state of mind, which is acting to protect a person, plan, behavior or belief. Mental Shield is automatically and naturally activated by the mind whenever an individual feels “absolutely sure” that the individuals can't (or won't) have something that they might desire. They act to make us as comfortable as possible without the things they feel they can't (or won't) have.

According to the present invention, a method of activating and using Mental Shields is named “The Shield Method.” In order to help to demonstrate the Shield Method, the term “Shield” is used in various ways as nouns, verbs, and etc., like “Shielder,” “Shielded” and “Shielding.” For example, the people who learn and use the Shield Method are referred as “Shielders”, and when a Shielder activates a Mental Shield, the individual is referred as “Shielded”. To distinguish these terminologies, the first letters of all words relating to “Shielding” in the present invention are capitalized.

Obviously, it is much easier to resist a temptation when the powerful mind of an individual is programmed to help him or her or her to resist it than when it is working for the temptation. It's important to note that this sure-programmed state of mind is not unusual or stressful. It's naturally and comfortably activated in humans all the time, such as when people goes to sample tables, or get dressed before going out into public (they feel sure that they must go to the trouble of getting dressed before going out, which programs their minds to help them to do it, as comfortably as possible, and they succeed over 99% of the time.)

“Mental Shielding” is a condition of a mind that the mind naturally uses to make an individual comfortable when he or she feels sure that he or she can't have something that he or she might desire. It also enables people to accomplish tasks and goals that require suppression of their primitive impulses. It provides much greater ease, comfort and reliability than any mandatory method of personal control, so it's highly desirable to be able to activate and achieve it reliably and “at will”. The promises-tracking device and method thereof according to the present invention are provided to enable a participant to activate his or her mental shielding and control his or her personal behavior, especially in personal situations at will. Conventional mandatory methods, such as diets, cause stress and typically only achieve success rates of less than 3%. The activation of Mental Shielding of the present invention, however, typically makes resisting temptations easy and comfortable and gives success rates greater than 99%.

The question is how to activate and keep the mind of an individual in mental shielding state. The present invention provides a promises-tracking device and a method thereof for enhancement of behavior, wherein the promises-tracking device is embodied as a program operated electronic device configured to form a promise chain and a promise score for tracking promises of a participant, enabling the participant in a “Mental Shielding” state to resist temptations.

Generally, people are often able to create this sureness when they are motivated to do something. The problem is that they usually can't maintain it. For example, when most people start a weight loss or exercise plan, they are highly motivated and able to manufacture a sure state of mind that they will follow. This programs their minds to help them to follow their plans, at first. But when the behavior becomes unpleasant, the primitive mind of an individual will start fighting his or her plan, giving urges, rationalizations, etc. to stop it. Then they will have to fight their powerful primitive minds, using ordinary wavering willpower, to try to stick to their plans.

The present invention provides a promises-tracking device and a method thereof to help people create a new reliable superego influence, so that they can activate the Mental Shielding ability of their minds “at will.”

It is the “social” superego influence that enables the over-eater at the sample table to feel sure that he can't take more than one sample, which programs his mind to help him or her resist taking more. The “new superego influence” according to the present invention is created by establishing a commitment to a “chain of promises” through the promises-tracking device of the present invention, so that once the individual adds a promise to his or her promise chain, he or she will feel sure that he or she will keep it with a high degree of reliability. Then, whenever he or she adds a promise to his or her promise chain, he or she will be able to activate the Mental Shielding capability of his or her mind with a high degree of reliability, which will program his or her mind to help him or her or her to keep his or her promises.

It is worth mentioning that the new superego influence is a circle. An individual must create a “mental sureness” that he or she will follow the behavior that he or she wants to program his or her mind to help him or her or her to follow, that will enable him or her or her to activate this sure state of mind “at will” even in personal situations, whenever he or she adds a promise to his or her promise chain, as long as he or she is committed to keeping his or her promises. For example, the over-eater will be able to program his or her mind to help him or her or her to resist temptations in his or her own home or wherever he or she is, so that he or she can easily, comfortably and reliably resist his or her temptations without having to fight his or her mind. In addition, according to the present invention, a further step is taken that not only will the participant, such as the over-eater, not have to fight his or her powerful mind to keep his or her promises, he or she will have all that power programmed to help him or her or her to keep his or her promises.

Of course, proving a mental state of sureness can be difficult. The invention does not attempt to prove this but, rather, to teach, assist and motivate people in achieving and maintaining a high level of sureness regarding keeping the promises the participant adds to his or her promise chain of promises. The Shield Method of the present invention helps the participant establish his or her chain of promises and make a strong commitment to keeping the promises he or she adds to the promise chain. The promises-tracking device of the present invention also teaches the participants techniques for using the resulting reliable control.

It is important to note that commitments are a normal part of life. People easily and naturally make commitments throughout their lifetime. They just decide that they want the benefits of something that requires a commitment enough to make the commitment. Commitments are easily and comfortably maintained, as long as the behavior is easy enough to follow and the benefits to them are sufficient to maintain them. Examples include the commitment to a spouse and to maintain the relationship, the commitment to drive in the legally designated lane and avoid getting in auto accidents, and simple commitments such as always adding detergent to loads of wash and to make sure the clothes get clean.

The promises-tracking device and the method thereof of the present invention are provided for assisting the participants to make a chain of promises with specific steps so as to help people make a very strong commitment to keeping the promises they add to their chains, so that they can reliably achieve the sureness required to program their minds to help them keep them.

The promise chain established according to the present invention significantly makes all the difference. Without the chain of promises of the present invention, there are not sufficient consequences to create the required sureness to program the mind. If the participant just makes a single promise, such as the over-eater does one bad thing regarding his or her diet, it is just one misdeed that he or she can easily rationalize that he or she can make up for “tomorrow.” For example, when tempted to break his or her diet today, he or she can simply tell himself or herself that he or she will make up for it by fasting tomorrow. Therefore, he or she can always rationalize that there will be no consequences whatsoever. In fact, every “tomorrow” he or she can make another “procrastination rationalization”.

However, by linking every promise to a promise chain that gives him or her or her easy, comfortable, reliable personal control and won't give him or her or her these benefits if he or she does not keep his promises consistently. That is a sufficient consequence to help him or her or her remain sure that he or she must keep his or her promises consistently.

According to the preferred embodiment of the present invention, a promises-tracking device, which is a program operated electronic device 1 configured to form a promise chain and a promise score for tracking promises of a participant, comprises:
at least one processor 10;
an input device 11 comprising a time setter 111, an activator 112, an excused broken promise button 113, and an unexcused broken promise button 114;
at least one memory module 12 stored program to instruct the at least one processor 10 to receive a plurality of viable promises inputted by the participant through the input device 11 in a chainlike manner and store the plurality of viable promises to form the promise chain on the at least one memory module 12; and
a screen 13 configured to display a physical activation signal generated through the activator 112 of the input device 11 to notify while the viable promises is sealed in the promise chain, wherein setting promise times in response to the viable promises of the promise chain in a numbering order in the memory module are set via the timer setter 111, such that a notification when the promise times are up is displayed via the screen 12.

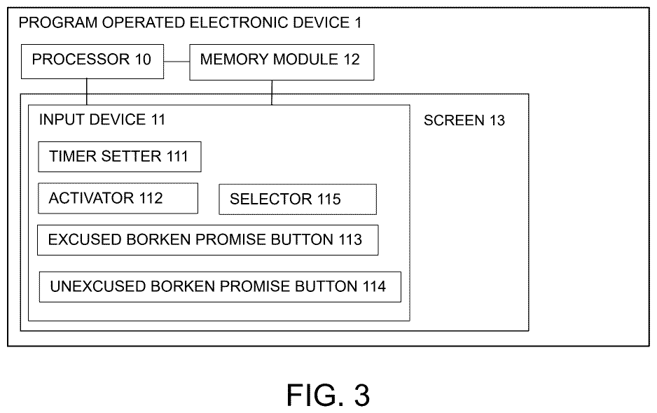

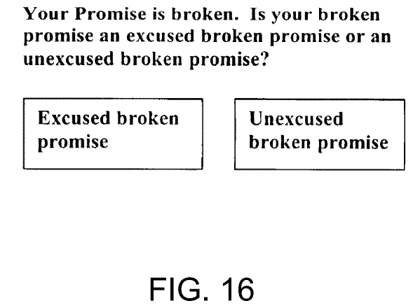

According to the preferred embodiment, the excused broken promise button 113 (as shown in FIG. 3 and FIG. 16) and the unexcused broken promise button 114 (as shown in FIG. 3 and FIG. 16) are configured to be selectively inputted for an input of a broken promise when one of the viable promises is broken that the promise chain is then broken to have a negative effect on the promise score of the participant which is determined and generated regarding the promise chain and displayed via the screen 13, wherein a level of sureness of the participant is displayed via the screen graphically for the participant to view and periodically for monitoring the level of the sureness of the participant.

The at least one memory module 12 is configured to further instruct the at least one processor 10 to determine whether the one of the viable promises being broken is the excused broken promise or the unexcused broken promise by the at least one processor 10, wherein only the unexcused broken promise has the negative effect on the promise score. The excused broken promise and the unexcused broken promise are selected by the participant via the input device 11.

According to the preferred embodiment, the program operated electronic device 1 further comprises a selector and a baseline documentation. The selector is configured to enable the participant to have a selection of a learning and training phase with one or more lessons for techniques of effective promise stored in the memory module according to a personal control score of the participant. The baseline documentation, which is stored in the memory module and displayed, via the screen, includes a plurality of personal, health and habitual questions for the participant to answer in order to obtain a personal control score for evaluating what kind of lesson and assignment in the learning and training phase to be displayed to the participant, wherein lesson material and suggestions are selected to the participant according to the personal control score through the selector, so as to help the participant to restore the sureness of the participant when the level of sureness drops.

It is worth mentioning that the promise score contains information selected from the group consisting of total promises added to the promise chain, total broken promise, and percentage of successful promise.

According to the preferred embodiment of the present invention, referring to FIG. 1 and FIG. 3, the program operated electronic device 1 can be embodied as a smart device, which is programmed with an application, comprising the processor 10, the at least one memory module 12 and a touch screen as the input device 11. The home key of the smart device can be embodied as the activator 112 while the touch screen is programmed to show a window as the screen 13 and a plurality of icons, including a first icon as the time setter 111, a second icon as the excused broken promise button 113 (as shown in FIG. 3 and FIG. 16) and a third icon as the unexcused broken promise button 114 (as shown in FIG. 3 and FIG. 16) configured to be responsive to a touch by the participant.

According to the preferred embodiment of the present invention, referring to FIG. 2 and FIG. 3, the program operated electronic device 1 can be embodied as a tablet computer, which is programmed with an application, comprising the processor 10, the at least one memory module 12 and a touch screen as the input device 11, which is programmed to show a window as the screen 13 and a plurality of icons, including a first icon as the time setter 111, a second icon as the activator 112, a third icon as the excused broken promise button 113 (as shown in FIG. 3 and FIG. 16), and a third icon as the unexcused broken promise button 114 (as shown in FIG. 3 and FIG. 16) configured to be responsive to a touch by the participant.

According to the preferred embodiment of the present invention, referring to FIG. 1 to FIG. 4, the Shield Method is embodied as a promises-tracking method for enhancement of behavior of the participant through the program operated electronic device 1 configured to form the promise chain and the promise score for tracking promises of the participant, which comprises steps of:
(a) recording a plurality of viable promises made by the participant in a chainlike manner through the input device 11 of the program operated electronic device 1 to form the promise chain and storing the plurality of viable promises of the promise chain on the memory module 12 of the program operated electronic device 1;
(b) generating a physical activation signal through the activator 112 of the program operated electronic device 1 to notify while the viable promises is sealed in the promise chain;
(c) in response to setting promise times of the viable promises by the participant via the time setter 111 of the input device of the program operated electronic device 1, numbering the viable promises in an order in the memory module 12;
(d) selectively notifying the participant via the program operated electronic device 1 when the promise times are up;
(e) determining and generating a promise score of the participant regarding the promise chain, which is an incentive to motivate the participant to keep the promise score as high as possible, wherein when one of the viable promises is broken, the promise chain is then broken;
(f) determining whether one of the viable promises being broken is an excused broken promise or an unexcused broken promise by the program operated electronic device, wherein only the unexcused broken promise has the negative effect on the promise score, wherein the excused broken promise and the unexcused broken promise are selected by the participant via the input device 11 of the program operated electronic device 1; and
(g) providing a level of sureness of the participant on the screen 13, wherein the level of sureness of the participant is indicated graphically on the screen 13 of the program operated electronic device 1 for the participant to view and displayed periodically on the screen 13 of the program operated electronic device 1 for monitoring the level of sureness of the participant.

The step (e) further comprises steps of:
(e1) receiving an input of a broken promise selectively by the participant via one of the excused broken promise button 113 and the unexcused broken promise button 114 of the input device 11 of the program operated electronic device 1 when one of the viable promises is broken that the promise chain is then broken to have a negative effect on the promise score, and
(e2) displaying the promise score on the screen 13 of the program operated electronic device 1.

According to the preferred embodiment of the present invention, in responsive to a selection of the participant through a selector 115 provided on the input device 11 of the program operated electronic device 1, the program operated electronic device 1 provides at least a learning and training phase with one or more lessons for techniques of effective promise stored in the memory module 12 to the participant according to a personal control score of the participant, wherein a baseline documentation phase is provided and stored in the memory module 12, wherein a plurality of personal, health and habitual questions are given for the participant to answer in order to obtain a personal control score for evaluating what kind of lesson and assignment in a learning and training phase to be given to the participant, wherein lesson material and suggestions are selected to the participant according to the personal control score through the selector, wherein the lesson material and suggestions are provided to the participant via the screen 13 of the program operated electronic device 1 to help the participant to restore the sureness of the participant when the level of sureness drops. Correspondingly, the promises-tracking method further comprises a step of:
providing a learning and training phase by the program operated electronic device to the participant, wherein in the learning and training phase, one or more lessons are given to the participant regarding effective promise techniques, and one or more assignments for the participant to complete regarding the promise techniques, wherein an assignment score of the participant is determined regarding a completeness of the assignment.

It is appreciated that, instead of or in addition to receiving the learning and training phase provided by the program operated electronic device 1, the participant can of course come into an office or clinic for appointments and learn through the use of lessons and support group meetings for the additional support that the present invention provides. However, the program could be taught in almost any other format: book, home-study course, computer program, video or audio tapes, seminar, etc. Support is highly recommended and could also be given in many other ways, such as by interactive computer program, telephone calls, scheduled messages, FAX, on-line support groups, meetings, computerized or manual support system, etc.

The step (g) further comprises a step of:
(g1) monitoring the level of sureness of the participant for ensuring the participant is able to keep the viable promises, wherein when the level of sureness drops, lesson material and suggestions are provided to help restore the sureness of the participant.

In the step (f), the excused broken promise and the unexcused broken promise are selected by the participant. In the step (e), the promise score contains information selected from the group consisting of total promises added the promise chain, total broken promise and a percentage of successful promise.

The assignment score contain information selected from the group consisting of total number of assignments given, total number of assignments completed satisfactorily, percentage of assignments completed satisfactorily, number of retake assignments given, number of retake assignments completed satisfactorily, and a percentage of retake assignments satisfactorily completed.

The participant is allowed to retake the assignment and a “panic button” is provided on the input device 11 of the program operated electronic device 1 for the participant to access that results in lesson material, tips and suggestions to be provided for helping restore the confidence of the participant.

According to the preferred embodiment, the baseline documentation phase is preferred to execute prior to starting the learning and training phase, wherein in the baseline documentation phase, a plurality of personal, health, and habitual questions are given for the participant to answer in order to obtain the personal control score for evaluating what kind of lesson and assignment are given in the learning and training phase.

In addition, the promises-tracking method further comprises a step of:
providing an introductory phase prior to starting the baseline documentation phase, wherein in the introductory phase, information is provided for helping the participant establish the promise chain, for teaching the participant how to make promise techniques, and for guiding and motivating the participant to succeed.

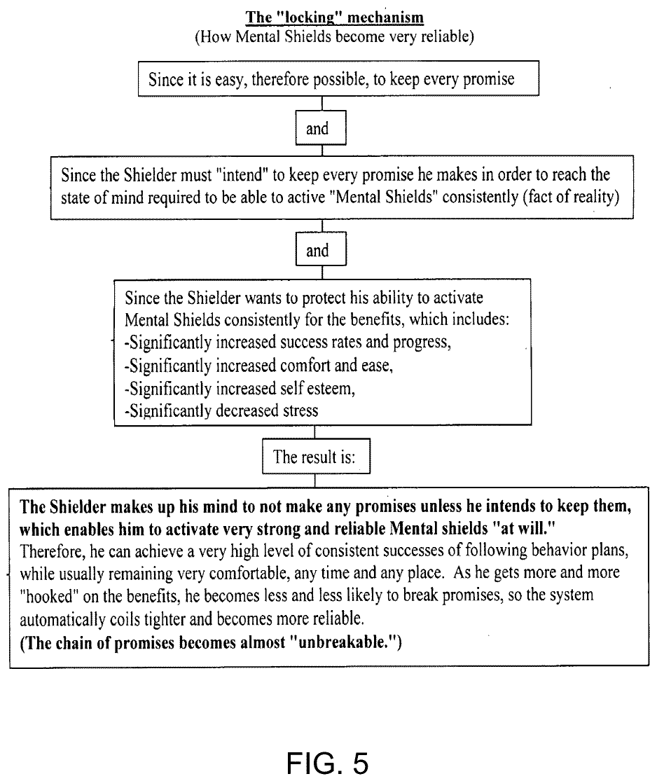

Referring to FIG. 5, a locking mechanism of the promises-tracking method according to the present invention is illustrated. The Shielder participant, is very to follows the steps to keep the promises the participant adds to his or her promise chain. By selection of the excused broken promise button 113 or the unexcused broken promise button 114, the participant is even allowed and excused for forgetting to keep them. Accordingly, the Shielder participant highly values the personal control that results from being to program his mind to help him or her keep his promises. Practically, the consequence of losing this precious control is strong enough to keep the Shielder participant comfortably committed to keeping his or her promises.

According to the clinical test results, the chain of promises has a doable, lasting quality that can finally make it possible for people to succeed at weight loss, wherein all participants of the test group said that they could easily maintain their control indefinitely, even those who gave up previously on diet after diet. There is a significant improvement.

It is worth mentioning that the possibility to have an “id” desire must be completely eliminated in a person's mind for their ego to be able to form and maintain a reliable Mental Shield. This is why it was never possible to activate the “Shielding” ability of the mind for weight loss (and other personal control applications) with the conventional methods. How could the possibility to have excess food be eliminated, when food is everywhere around us or so easily obtainable in today's society? The present invention is the first that eliminates the real possibility to have desires in the person's mind. For the first time, this Mental Shielding ability can enable the Shielder participants to comfortably and successfully “pass up” short-term desires, such as tempting foods, so that they can achieve their long-term desires, such as weight loss.

It should be noted that there has been some confusion about the “absolute sureness” as mentioned above. This happens at the moment a person “decides” to keep (or go ahead with the behavior of) his promise. It does not mean that the individual is always sure that he will keep every promise, any more than most people are always sure that they will go to work every day. The excused or unexcused broken promise determining step (f) through the excused broken promise button 113 and the unexcused broken promise button 114 of the promises-tracking device of the present invention significantly solves such mental problem and enables the Shielder participant to enhance his or her behavior control. With a strong commitment to keeping his promises, the Shielder participant will usually feel sure that he or she will keep them; but his sureness can occasionally waver and may only become absolute seconds before deciding to keep a promise.

The decisions of adding promises to the promise chain of the Shielder participant and the execution actions of the excused broken promise button 113 or the unexcused broken promise button 114 even a promise is broken can activate Mental Shields, although it takes sufficient consequences to maintain them. It's natural and healthy to occasionally “flirt” with ideas of doing other than what we feel we should do, to weigh the consequences. But once the Shielder participant becomes “sure” that he or she has too much to lose to break his or her chain, and decides to keep the promise, his or her mind will be programmed to assist with the behavior of the promise rather than fight it, since sureness programs the mind.

Once a Shielder participant is fully committed to keeping the promises he or she adds to the promise chain thereof, he or she has achieved the primary Shield Method goal and benefit of reliable personal control. Then he or she is taught the “promise techniques,” for using the leverage of this reliable control to make effective promises that can help him or her or her achieve his or her goals.

As shown in FIG. 5, by processing the steps of the promises-tracking method with the promises-tracking device of the present invention, the Shielder participant intents to keep every viable promise he or she makes since, per each viable promise the Shielder participant added in his or her promise chain, the Shielder participant is required to execute an action, such as actuating the activator 112 for once to notify the program operated electronic device. In other words, it is not a mind state of thinking to make promise but a physical action taken by the Shielder participant to add a viable promise to the promise chain.

In addition, the timer setter 111 helps the Shielder participant to set a duration time for each promise and, in the step (d), the promise time illustrated in the screen 13 informs the Shielder participant the promise time is up and the participant has successfully kept the promise. After each promise in order to reach the state of mind required to be able to active “Mental Shields” consistently. The step (b) of generating the physical activation signal through the activator 112 of the program operated electronic device 1 ensures the Shielder participant making up his or her mind to not make any promises unless he intends to keep them, which enables him or her to activate very strong and reliable Mental Shields “at will”. Therefore, the Shielder participant can achieve a very high level of consistent successes of following behavior plans, while using remaining very comfortable, any time and any place. As the Shielder participant gets more and more “hooked” in the benefits, he or she becomes less and less likely to break promises, rendering the Shield method with the promises-tracking method automatically coiling tighter and becoming mor reliable.

Many of these techniques are specifically aimed at reversing the activities of the primitive id division of the mind. A number of techniques to accomplish this involve making promises to “delay quantity.” The timer setter 111 of the program operated electronic device 1 of the present invention is configured to achieve such objective. This acts in direct opposition to id urges for immediate quantity (instant gratification). With one such technique, for example, an individual promises to eat half of his usual portion and then wait 15 minutes before eating more. This is an effective technique since it stops the “hand-to-mouth” reflex and allows time for the food he eats to register feelings of fullness and satisfaction. A second part of this technique is to get busy doing other things (rather than sitting and watching the clock) during the 15 minutes. This helps trick the mind into feeling that he is done eating. If he still wants more after the 15 minutes, a technique is to promise to have “just a little more” for another 15 minutes.

It should be noted that keeping the promises of these techniques will only be easy and comfortable when a person has a strong commitment to his chain of promises, so that his or her mind is programmed to help him or her or her keep them. Otherwise, his or her powerful primitive mind can fight his promises, making it extremely difficult and unpleasant to keep them. For example, trying to eat just one cracker can be difficult and feel like unbearable deprivation to the over eater, unless his or her mind is programmed for this behavior by means of the present invention.

The steps of the promises-tracking method of the present invention executed in personal situation by the Shielder participant having made a strong commitment to keeping the promises the Shielder participant adds to his or her promise chain makes the Shielder participant's promise chain as unbreakable as other “chains of behaviors” that he is committed to, such as to cover all of the checks he or she writes without intentional exceptions; to make every house payment without intentional exceptions; and to add detergent to every load of wash without intentional exceptions. The present invention gives the Shielder participant reliable personal control any time he or she adds a promise to his or her promise chain—even in personal situations that he could not reliably control before.

For example, when fat Albert is sitting in his living room and has a desire for some pie in his kitchen, he can now add a promise to his promise chain, such as to have only one small piece for three hours. Then Albert is required to generate a physical activation signal through the activator 12 of the program operated electronic device 1 in the step (b) and set the timer setter 111 for the three hours in the step (c). Accordingly, since he is surely committed to keeping his chain of promises, these actions taken will program his mind to help him or her feel comfortable with just that much. Should he want more after the three hours, he is completely free to have it and reset the timer setter 111, i.e. relieving all pressure. But he always has the ability to add promises to his promise chain, knowing powerful promise techniques to use to reverse the impulses of his primitive mind, to help him or her easily and comfortably stay in control. His self-esteem from his ability to reliably control his behaviors—and from the resulting weight loss—will naturally soar.

Practically, each promise Albert makes “locks-in” better behavior. Any promise to do better is an improvement. Making a lot of little improvement promises can quickly add up to real progress towards his weight goal. By the Shield Method of the present invention, many ideas for making it very easy are provided, yet very effective promises for reliable and consistent progress towards his weight goal. He can make promises for any eating improvements that he can think of—the possibilities are nearly limitless.

The promises-tracking method of the present invention is also a business method for teaching the Shield Method and providing enhancing support. It can decrease the number and severity of temptations and urges to do other than a person's specifically planned behavior intentions. In addition to enabling people to withstand strong temptations and urges to do other than their specifically planned behavior intentions, it can make it easier for them to resist mild to moderate temptations and urges to do so. It can significantly reduce the need to expend energies required to maintain willpower. It can make people much more comfortable while performing specifically planned behaviors intentions. It can significantly increase people's ability to control their behaviors. It can significantly increase people's confidence in their ability to control their behaviors and achieve their goals. It can significantly increase people's self-esteem and sense of accomplishment. It can make it easier for people to resist strong temptations and urges to do other than their specifically planned behavior intentions. It can be relied upon to work almost any time and any place. It can deflect urges and temptations to do other than a person's specifically planned behavior intentions, so that they are decreased or eliminated. It can enable people to be able to quickly regain control if they start to lose it, such as when a binge begins. It can improve habits, even permanently, wherein the present invention is the first method to date that can help a majority of people easily and quickly establish new habits, and it may be the first true cure for “Binge Eating Disorder” (B.E.D.). It can significantly increase an individual's ability to succeed at personal goals, while also significantly decreasing their levels of stress. It can help establish new “better” habits that are preferred over old “bad” habits. Many methods attempt to teach better behaviors, but these may not be preferred. It can improve health by helping people achieve goals in areas of weight loss, exercise, control of alcohol consumption, etc. It can be used to control almost any behavior, including “do” behaviors, such as “do exercise” as well as “don't” behaviors, such as “don't” eat a second helping. It provides the promises-tracking method for obtaining personal goals that not only reduces stress, but is as enjoyable, exhilarating and fun as a game, enabling people to be able to stay on it indefinitely for consistent progress. It can often eliminate the need for risky, invasive methods of personal control. It significantly reduces major setbacks in progress. It can enable many weight loss programs that have not worked to work now.

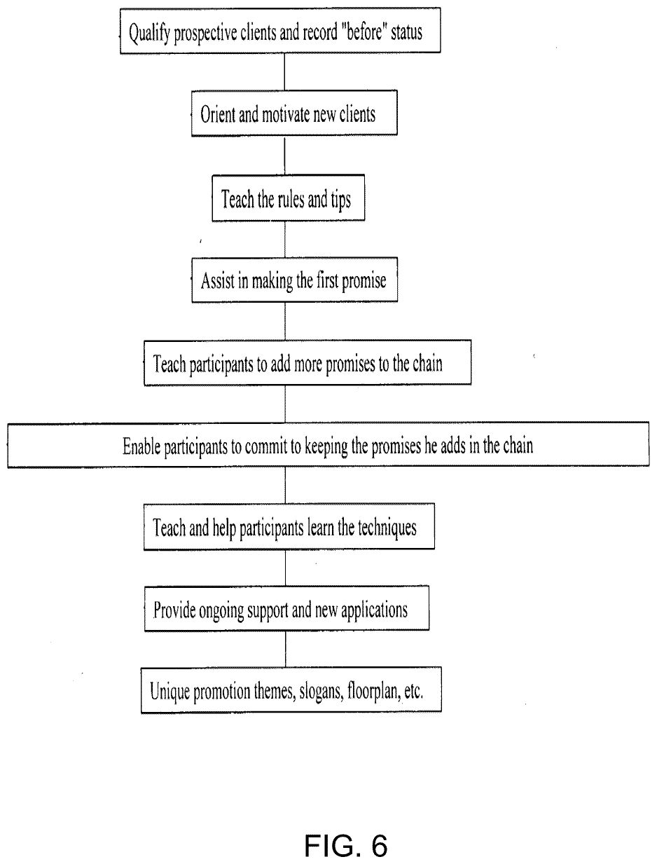

In the baseline documentation phase and the learning and training phase, as shown in FIG. 6, the following steps (not required to be given in the exact order) teaching the participants to create and use the chain of promises:
Step 1: Qualify prospective participants and record “before” status. This is accomplished by collecting information about their current habits, current levels of control and motivation and goals. If they are found to be good candidates, payment arrangements are made. A more detailed “before status” analysis (of current habits, goals, physical status, etc.) may be then completed before proceeding to the next step.
Step 2: Orient and motivate new clients. New participants are taught for the Shield Method terminology, such as what Mental Shields are, more specifically, and the benefits that the Shield Method can offer them. The participants are informed of what to expect and what is required of them, regarding attendance, etc. A preferred embodiment is weekly individual learning and evaluation appointments, for example through the program operated electronic device or come into the office or clinic, followed by weekly support group meetings, as needed or desired. New participants are preferred to be verified to understand essential information by testing or through the use of an interactive computerized format. The following is an example of an orientation description that may be provided:

What are Mental Shields? A Mental Shield is a programmed state of mind, which is acting to protect a person, plan, behavior or belief. Mental Shields are activated by the mind whenever we feel “absolutely sure” that we can't (or won't) have something that we might desire. They make us comfortable without the things we feel we can't (or won't) have. For example, when we are in a restaurant waiting for a table and see food on a stranger's plate (who is currently eating it), we feel sure that we can't walk over and take the stranger's food—even if the food is very appealing and we are quite hungry. This is the mental state required to activate a Mental Shield, so we are “Shielded.”

When Shielded, our mind won't work to obtain that specific food on the stranger's plate (although it may inspire us to order the same thing). Rather, it will be programmed to help us remain comfortable while waiting to be seated and served. The mind is so good at this that it even makes us sort of “blind” to the stranger's food (we can see it but in a different, non-pursual way). This example illustrates the high success and comfort rates of Mental Shields. They are usually more than 99% effective at protecting behaviors while keeping us as comfortable as possible.

This is an example of a permanent “Social Shield,” not a “Personal Behavior Shield” (PBS) that the Shield Method teaches people to activate. When an over-eater is home, they're likely to eat anything and everything they want because they are not protected by a Behavior Shield at home.

The Shield Method enables people to activate very reliable, very strong Personal Behavior Shields “at will.” This can give them very comfortable and reliable personal control at home or anywhere else. This is why I named this method the “Shield Method,” because it “Shields” or protects people from the discomfort of having to fight their minds and the resulting failure, low self-esteem, etc. It also protects their success in tempting situations.

Learning the Shield Method is like learning the rules and techniques of a game. The ordinary steps produce an utterly unexpected, extraordinary result. The individual follows the rules and they work instantly, without having to practice, although practicing the techniques of using them will increase their skill and ease of obtaining goals. In a preferred embodiment, Shielder participants learn by attending individual appointments and support-group meetings. Time is allowed between appointments for practice and to let the information sink in. The Shield Method can dramatically improve personal control and can be used to obtain many personal goals, such as weight loss and to exercise consistently without a personal trainer, etc.

To orient the individual to the concept of “Mental Shielding,” scientific descriptions and examples may be given, such as those given in this application.

For example, “mental Shielding” may be described in the psychological terms of the id, the ego and the superego, the three divisions of the mind or “psyche.” The id is the primitive mind (instinctual drives). The superego represents the moral standards and ethics that you believe in. It rewards you with good feelings about yourself when you meet these standards, and makes you feel bad when you don't.

The ego mediates between the above 2 divisions of the mind. According to Taber's Encyclopedic Medical Dictionary, “The ego possesses consciousness and memory and serves to mediate between the primitive instinctual or animal drives (the id), internal social prohibitions (the superego), and reality. Thus, the ego allows one to adapt to what might otherwise be a very unpleasant situation. The psychiatric use of the term should not be confused with its common usage in the sense of ‘self-love’ or ‘selfishness’.

To clarify, think of the above example of being in a restaurant: When you are very hungry and want to eat (a primitive id desire), but feel “absolutely sure” that you can't take a stranger's food (because of a strong social superego influence), your ego will form a Mental Shield to protect you from the id desire, so that it is very easy and comfortable for you to be able to resist the stranger's food. If the mind couldn't do this, we couldn't adapt when it is not possible to have things that we want, and this would be “very unpleasant,” as the above Tabor's definition describes.

The Shield Method of the present invention enables people to create a new, very strong superego influence, so that they can activate the natural “Shielding” ability of their egos. Or it may be the ego activating a protective mechanism as it does whenever a person cannot have a desire. Regardless, whenever a person attains the required state of mind, we have noticed that a Mental Shield always forms, in keeping with the Scientific Method. (Except in neuroses, when instinctual drives are in conflict with those dictated by the superego.)

The action and benefit of Mental Shields are that they actually “reverse” the activity of the mind. They stop the mind from working “for” desires that we feel we can't have; and they put the mind to work to help us succeed at resisting them. As in the above example, when we feel that can't take food from a stranger's plate (even though we could), a protective Mental Shield will be activated through executing the steps of the promises-tracking method of the invention. Then our mind will not work to send ideas, thoughts, plans, rationalizations, etc. into our consciousness of taking the other person's food. Instead, it will do everything in its power to suppress these thoughts and help make us comfortable without the other person's food. It's so good at this that it is as if we are “blind” to the other person's food, in a pursual way. The actions of the mind to help us comfortably succeed can be mental, neuronal, chemical, physical and/or biological. As Pavlov's experiments with dogs, and countless other experiments have since proved, our minds affect all of these systems.

Mental Shields can be nearly absolute and unwavering, such as a parents unwavering commitment to care for a child; or a person's unwavering commitment to turn lights off before leaving a room. I refer to these kinds of Mental Shields as “Permanent Mental Shields.” However, even these Shields can be broken under certain circumstances. For example, under extreme circumstances, almost anyone can be provoked to commit murder.

Mental Shields are perfectly safe because they utilize the way the mind naturally works. In fact, “Naturally Thin Eaters” have permanent Mental Shields which enable them staying thin comfortably and successfully. The only time an over-eater can experience what this is like is when they start a diet. Then, a temporary Mental Shield usually “materializes,” and helps them sail by the donuts and other tempting foods without even pursually “seeing” them. But Diet Shields are only temporary. Sooner or later they disintegrate, and the dieter starts feeling uncomfortable and stressed because the id portion of the mind starts working for their habitual food desires. There has never been a way for dieters to maintain the state of mind that makes it easy and effortless before.

We all have and use Mental Shields in many areas of our lives (Credit Shields, Morality Shields, Social Shields, Physical Shields, etc.). Mental Shields are what allow us to act in civilized ways, rather than primitive ways. They are how we can enjoy eating in social situations, like restaurants, without fighting over food like packs of wild animals; and how we can live together in peace. They create safeguards for advanced societies so that we can protect the things that are important to us. They enable us to reach worthwhile goals that require us to work hard and pass up instant gratification.

When a person is not Shielded from a desire and their mind is working for it, we say that they are “Shielded for” the desire. When Shielded for a desire, a person may not listen to reason, because their mind will exaggerate the positive aspects of the desire and try to block out or selectively ignore the negatives. Sometimes only after the person obtains the desire will they be released from the Shield and able to clearly see the negative aspects of having the desire. As the saying goes, “Be careful what you wish for because you may get it and then realize you don't really want it.”

Mental Shields give incredible advantages and should be highly valued and protected because they allow us to surely and easily resist primitive desires. For example, a person with an Honesty Shield in a specific situation will be able to effortlessly return a wallet full of money that they find. It can be mentally painful and even impossible for a person without an Honesty Shield for the same situation to resist the desire to keep the money. We all have a “Shield threshold” in the amount of pressure our Mental Shields can take. For example, under certain circumstances, almost any one's Morality Shield can be strained to the breaking point and they will commit murder (called “breaking” a Shield). Shields can be permanent or temporary.

We have not been able to create the state of mind required to activate Personal Shields “at will” before. We have had to live with whatever Personal Shields our parents or life experiences gave us, although the traditional art describes that it is very difficult to change the contents of our superego so that we can permanently change habits. The Shield Method finally gives us a way to activate Mental Shields “at will” and even help to develop new permanent Mental Shields that we may desire.
Step 3: Teach the rules and tips. In this step, the participants are prepared to form their chain of promises by teaching them the rules for making promises as follows:

Rules and Tips for Adding Behavior Viable Promises to the Promise Chain:

Viable promises must be easy, safe and short-term, preferably from less than 1 minute to no more than 6 hours in the beginning. This is to make it easy for the Shielder participants to keep their promises.

The Shielder participant is instructed in how to make viable promises that are “easy” for him or her or her to keep. This is no different than developing many other kinds of skills, such as ice skating. A new skater doesn't recklessly push himself onto the ice. He starts out cautiously, getting a feel for the ice, and stays within his level of ability. Similarly, the participant of the promises-tracking device 1 of the invention is required to make promises that will be extremely easy for him or her or her to keep, at first; and is given suggestions for making easy promises, such as promising to only have half of a roll for 30 minutes. As the ice skater uses feedback from his experience, and has a healthy “fear” of overdoing it, so does the Shielder participant, who gets a feel for making promises, and knows that he could “ruin his Promise Score,” or even fail if he overdoes it. This helps the Shielder participant stay within his or her level of comfort, in the same way.

Examples of very easy, yet effective—and even fun—promises to use until he gains greater skill and confidence are provided, such as promises to “lock in” his or her intentions. If he only intends to have one small piece of pie, he or she is encouraged to add a promise to his or her chain to only have that much for a certain amount of time, rather than leaving himself or herself vulnerable to another piece right afterwards. The Shielder participant is taught with the “3 more bites” technique, that is when he can't stop eating from a bag of chips, for example, he can make a promise to stop after 3 more bites—and it's all right to pick out the 3 biggest chips.

The Shielder participant may be encouraged to try to make a certain number of promises per day, such as 12 through the program operated electronic device (the promises-tracking device), which can easily be done by making three promises at every meal and three between meals. This can cut hundreds of calories a day—and Shielder participants are thrilled with their easy, comfortable control.

The Shielder participant is encouraged that the “Shielder” to start at his or her own level. The Shielder participant may be taught the following: “For some of you, it will be easy to make advanced promises, such as to have no fries with your sandwich. You've just lacked the control. (But why not Shield to have a few fries, if you really want them—bet you can eat just one.) Some new Shielder participants will not be able to make such advanced promises yet. Don't worry. Just make promises on your own level, or those that are comfortable for you. Maybe you can promise to have half of the fries. Or perhaps you can promise to have a better choice, such as a salad. If you can't make any of these promises, simply toss a few fries in the trash and promise not to get them back out. Get the idea? Making promises at your own level makes it easy for anyone to make at least 12 promises a day. You may notice that sometimes you will feel stronger than at other times, and you will be able to lock in better promises during those times. As you become more comfortable making and keeping promises, it will automatically be-come easier to make more difficult ones.”

Other techniques for making easy promises are provided by the promises-tracking device of the invention. For example, the Shielder participant is taught that reducing the term of a promise will make it easier to keep. Promising to have a small portion of something for 15 minutes is much easier than promising a small portion for 6 hours, and that short-term promises actually work better, since the primitive mind doesn't put up as much resistance to making them. Yet the Shielder participants effectively stop the hand-to-mouth reflex and give food time to register feelings of fullness and satisfaction. Add to this a promise that the Shielder participant must not sit and watch the clock for those 15 minutes, but must get busy doing non-eating things, and he or she may forget to have more. If the Shielder participant still wants more, he or she is encouraged to have it—although the Shielder participant is requested to consider having just one or two more bites. This is often all he or she needs.

One rule stipulates that the promise the Shielder participant adds must be short-term, preferably less than 6 hours. It is explained that promises have a length of time assigned to them, but they don't have to have an exact time. For example, if the Shielder participant is standing at a checkout counter at a grocery store, he or she can promise not to eat anything from his or her grocery bags until he or she gets home and puts the food away. It is found that short-term promises can be the most effective promises since they are easier to make, yet the Shielder participants stop the hand-to-mouth reflex and make it easy to stop eating. It is noted that longer-term promises may be allowed for certain behaviors, such as promising to attend appointments, and etc.

Another rule to only make promises to do “better.” For example, Shielder participants can't make promises to eat more than they usually eat.

Shielder participants can make promises at any time, more than one promises at a time, extend promises, make overlapping promises and can upgrade promises. For example, an upgraded promise is promising to have no more than 3 cookies for an hour, and then upgrading the food to something more healthy of equal or lesser value.

Shielder participants are completely free to have more food after keeping their promises, unless an extended or overlapping promise is in effect. For example, if a Shielder participant promises not to eat anything for an hour, after the hour is over he or she is completely free to have more food if he or she wants, unless another promise is in effect. This is important because it relieves all pressure for the Shielder participant only has to keep the current promise and he or she is completely free. It is found that the techniques of the Shield Method are very effective in preventing Shielder participants from losing control after their promises are over; and they even reduce stress, since they know they can always use one of the promises techniques if they start to lose control. A Shielder participant is not bound to keep any promise that is or becomes unsafe or unhealthy to keep.

Another rule may state that “Shielder participants” can only make promises that they want to make. It should note that the teaching and training process of the preferred in-office embodiment of the Shield Method is very effective in motivating most Shielder participants to make many promises consistently.

It is worth mentioning that the Shielder participants don't have to make any promise, but once they do, they are responsible to keep them. If they break any promise, they have to pay the following penalties:
The immediate “rule” consequence of breaking a promise is loss of “Shielding privileges” for 24 hours (the forfeiture of the right to add any promises to his or her promise chain for 24 hours). The purpose of this rule is to give an immediate penalty and also to help the Shielder participant values the ability to use his or her Shield any time at will.

The long-term consequence of habitually breaking promises is that the Shielder participant will lose all benefits of the Shield Method, since his or her ability to activate Mental Shields will deteriorate, as shown in FIG. 6.

Shielder participants are not held responsible if they “innocently” break a promise, such as when they forget they had made a promise, although they must take steps to remember their promises if it happens often (this doesn't happen to most Shielder participants very often).

When a Shielder participant is in the “act” of “innocently” breaking a promise, he or she does not have to stop instantly. It is found that the mind is so strong that one can't just stop eating a cookie that he or she forgot he or she had promises not to eat and spit it out immediately when he or she suddenly realize that he or she wasn't supposed to have it. Few Mental Shields are so strong that the Shielder participant can withstand the full, infinite power of the mind instantaneously. The mind will “break” the Shield, making the person break the promise. So the Shielder participant is allowed to make a follow-up promise at times like this, such as “Just 1 or two more bites.” Without this rule, Shielder participants would unnecessarily and innocently break their chain of promises.

The Step 3 may come before or after this point. For example, the Shielder participant is guided to make his or her first promise(s) while learning the rules. The rules may be altered, according to what works best for each Shielder participant and each behavior goal. For example, it may be determined that a different length of promise is better for certain goals or custom penalties for breaking promises may be helpful for certain Shielder participant.
Step 4: Assist in making the first promise: To initiate the chain of promises of the Shielder participant, the Shielder participant is usually being “walk them through” making his or her first promise. A suitable promise for this is, “I promise not to eat anything for 15 minutes”, after asking them to start his or her chain at a time when he or she will not be eating. Alternatively, the Shielder participant is instructed to mentally decide on a first promise, in accordance with the above rules and any tips or suggestions that he or she has provided. The Shielder participant is preferred to be instructed to make the promise mentally and physically by generating the physical activation signal through the activator 112 of the program operated electronic device 1.

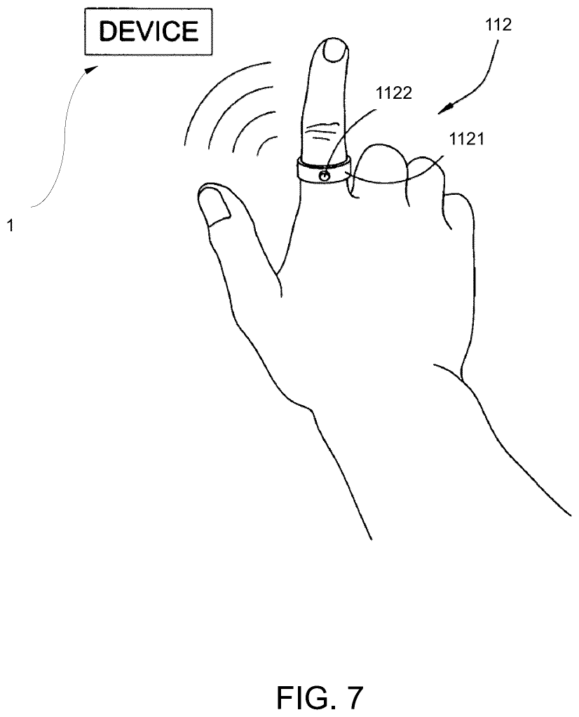

Then, the Shielder participants are instructed to use a “physical activation signal,” to start their promise and seal it to their promise chain. According to the preferred embodiment, the Shielder participant may execute the step (b) by generating the physical activation signal through the activator 112 of the program operated electronic device. Alternatively, as shown in FIG. 7, the activator 112 can be an independent element such as a ring 1121 with an activation button 1122 provided thereon and wirelessly linked with program operated electronic device 1, such that when the Shielder participant adds a promise to his or her promise chain stored in the memory module 12 of the program operated electronic device 1, he or she has no need to take out the program operated electronic device 1 but simply use one of his or her finger to physically touch or press the activation button 1122 of the independent activator 112 for a predetermined number of time. For example, with such finger ring type activator 112, when the Shielder participant decides on a promise, he or she may simply tap the thumb and index finger which wears the ring type activator 112 for 3 times to “seal” the promise to his or her chain of promises.

The Shielder participants are taught that a promise is not added to their promise chains until they physically seal it to their promise chains in this manner. Once it is added to their promise chain, they are responsible to keep it or pay the consequences (described below).

Incorporating the physical activation signal is essential, since it starts each promise and helps to distinguish between promises they just “thought about making” and promise they have “actually made.” It should be understood that other physical signals could also be used, but this physical act works particularly well for most people. It can be used very discretely any time and any place to “seal” additional promises to the chain. After a while, the signal may actually play a role in activating Mental Shields, since it is often the moment that the person becomes “absolutely sure” that they will keep their promise. However, it does not, in itself, activate a Shield. Only the state of mind of being “sure” that a person will keep his promise can activate and sustain a Mental Shield. By making his or her first promise, in this manner, the Shielder participant has initiated his or her chain of promises.

Each Shielder participant is taught that his or her goal is to feel “sure” that he or she will keep all of the promises he or she adds to his or her promise chain so that his or her mind will be programmed to help him or her or her keep them. Otherwise, his or her primitive mind will eventually begin to fight his or her promises, since they require that he or she pass up instant gratification. In addition, in order for beginners to achieve this sure state of mind, beginner Shielder participants just have to make up their minds to keep all of the promises they add to their promise chains, “no matter what.” The Shielder participant is reminded that it is very easy to keep his or her promises and encourage him or her or her to keep his or her mind made up to keep his or her promise so that he or she can experience what it feels like to be “Shielded.”
Step 5: Teach Shielder participant to add more promises to the promise chain. The Shielder participant is instructed to add more promises to his or her promise chain of behavior promises by following the above rules and tips of making promises that have given him or her or her. The Shielder participant is informed that his or her “Shielding ability” is not yet reliable; and assure him or her or her that he or she will later learn how to make them very reliable. The reason that it is helpful to have the new “Shielder” make unreliable Shields at first is that it helps him or her or her experience the benefits of Shielding so that he or she can value these benefits. This will enable him or her or her to complete the process of making his Shielding ability very reliable, as explained in Step 6 below.

Since the promises are easy and since the Shielder participants are taught to make up their minds to keep them, most Shielder participants can achieve the required sure state of mind to activate Mental Shields most of the time, even during this training process. The Shielder participants are encouraged to pay attention to the times in which they feel sure that they will keep a promise, because this is when a Mental Shield is in effect, that is their minds are programmed to help them keep the promises. The Shielder participant are encouraged to notice the changes in how their minds work when they are “Shielded” as follows:
(a) The essential change is a very high success rate of keeping their promises, when they feel sure that they will keep them;
(b) Other changes include feeling much more comfortable while resisting food desires that they have promised to resist which would ordinarily be difficult for them to resist;
(c) Much greater ease of resisting them.
(d) Not having to fight their minds as much to resist them, wherein fewer urges from their mind to have what they have promised not to have; and
(e) Feelings associated with increased personal control, such as increased self-esteem.

It is worth mentioning that the rules that promises should be easy, short-term and voluntary were designed to decrease resistance from the primitive mind to making promises, and also, to keep the individual comfortable. However, a person may not always be extremely comfortable keeping his promises, just like people are not always extremely comfortable keeping other Mentally Shielded behaviors (like paying every bill or feeding their families every day). Sometimes beginner Shielder participants count the minutes until a promise is over. But the programmed mind does practically guarantee success at following the behavior while making the person “as comfortable as possible.” The “boost” from the programmed mind usually makes them extremely comfortable.

Even if a Shielder participant does occasionally regret making a promise, he or she only has to “stick it out” for a short time, and then he or she is completely released, which is much easier than having to stick to a traditional diet for days, weeks, months or years.

Incentive can be given to the beginner Shielder participants to make numerous promises, such as to experience what it feels like to be “Shielded,” and to make easy, fast progress towards their goals. Most are almost immediately delighted to find that they can now, suddenly, easily succeed at behaviors that they could not easily succeed at, or do at all, before. For example, they now have the control to eat less food, like half of a bag of chips or just one cookie, instead of a whole bag. They can also “seal in” their eating intentions and prevent foreseen eating disasters with promises, as we encourage in the Step 7 as described below, as shown FIG. 6. In this way, Shielder participants begin to enjoy the benefits of making easy, steady progress towards losing weight, by making promises to do just a “little” better than they usually do. The Shielder participants are encouraged to notice and appreciate these benefits.

Some of the best promises for beginner Shielder participants are:
(i) make 3 promise to cut food at every meal;
(ii) make 3 promises every day between meals; and
(iii) use the following two-step technique: “First, cut more than you think you can; and second, decrease the term of your promise until it is easy to make the promise.” For example, promising to have half of a brownie for 3 hours may be a very difficult promise for a beginner to make. But, by decreasing the term to 30 or even 15 minutes, suddenly it's easy. In addition, these promises can be very effective—especially if the Shielder participant adds to the promise to not to just sit and watch the clock, but to get busy doing other things for those 15-30 minutes.
Step 6: Enable Shielder participants to form very reliable Mental Shields by teaching the “Locking Mechanism”, as shown in FIG. 5. Up to this point, Shielder participants have been enjoying their ability to Shield, but the Mental Shields they are activating are not reliable. The Shielder participants are really only keeping their promises because they are easy to keep and because they “want” to. However, this may not enough because after the newness of this method wears off, their primitive mind will start working against their promises because they require them to pass up instant gratification that it will begin sending urges to have a second roll, for example, when they have promised to only have one.

Therefore, it is very important that the Shielder participants make a very strong and lasting commitment to keeping the promises they add to their promise chains, so that they can reach the state of mind required to activate Mental Shields when they add promises to their promise chains, with a high degree of reliability. It is accomplished by using any or all of the methods listed in the steps of this application, for example the Step 7 of “Outline of Lessons,” described below. The most important of these is the “Locking Mechanism” concept.

As shown in FIG. 5, there are 3 components of the Locking Mechanism that enable the Shielder participant to achieve and maintain the sureness that he or she will keep the promises he or she adds to his or her chain with a high degree of reliability.

First, it is easy, and therefore “possible” to keep every promise. Shielder participants are taught that it is not possible to “Shield” behaviors that are too difficult to follow. This is one reason why traditional diets break down. The complicated rules and restrictions of traditional diets are simply too difficult for most people to follow for very long. Urges for favorite foods and recipes—and to be free of restriction—can become as strong as urges for air. This is why their primitive mind works so hard to get people off of traditional diets. But the present invention is doable. The rules thereof are designed to make it easy to keep every promise the Shielder participant makes every day for the rest of his or her life. These rules even excuse them for innocently breaking a promise or if it's unsafe to keep one.

Second, it is a true, unchangeable fact of reality that Shielder participant “must” intend to keep every promise to be able to activate Mental Shields consistently. The Shielder participants are taught that although they don't have to make any promises; once they do they can't even “intend” to break them. The reason is that if a Shielder participant allows himself or herself to intend to break an occasional promise, then at every strong temptation he or she will have to ask himself or herself, “Is this one of the promises I will allow myself to break?” Therefore, he or she can never feel sure that he or she will keep “any” promise. In other words, he or she can never rely on achieving the state of mind required to activate Mental Shields. He or she cannot have both exceptions in keeping his or her promises and personal control.

Third, because the Shielder participant learns to highly value and desire the benefits that he or she has never had before for personal control applications, such as being able to significantly increase success rates at achieving goals with greatly enhanced comfort, ease, self-esteem and decreased stress, he or she is motivated to protect this ability.

As a result, Shielder participants can make up their minds not to make any promises unless they intend to keep them and therefore can enjoy all of the benefits of this invention that they value. As they get more and more “hooked” on the benefits, they become less and less likely to break promises. So, the present invention automatically becomes stronger and more reliable.

The Shielder participants are taught about “false” Mental Shields. With false Mental Shields, such as “Diet Mental Shields,” the person's mind will only help the follow the behavior as long as he wants and is motivated to follow it. As soon as it becomes unpleasant to follow and he no longer wants to follow it, his or her mind can “about face” and work against his or her diet. With “true” Mental Shields, the mind works to help the person follow the behavior even when it is difficult to.

It is worth mentioning that a person who intends to break promises will eventually not be able to activate Mental Shields at all, per our observations, because his or her “Promise Shield” will be a “false” Shield. His or her mind will only work to help him or her or her keep his or her promises when it's easy to do and he or she wants to. It will be free to work against his or her promises when it's difficult to keep them. Since he or she won't have the advantages and success rates that true Mental Shields provide, he or she won't be any better off than before he or she started his or her promise chain (he or she will have to use willpower to keep his or her promises). Intending to break promises also welcomes unwanted desires into the conscious mind.

The Shielder participants may be provided with analogies about similar “behavior chains” they already easily and comfortably maintain. For example, covering every check they write at a bank. If a person wants the benefits of having a checking account, they must be responsible to cover every check they write. In the same way, if they want the benefits of being able to activate Mental Shields, they must be responsible to keep the easy, short-term, voluntary promises that they make. Maintaining their chains of promises is no more stressful than their chains of bank checks. The rules even excuse them for making innocent mistakes, such as breaking a promise because they forgot, something that their banks won't allow. And it's important for them to “think twice” about their abilities to keep a promise before making it, just as it's important to “think twice” about their abilities to keep checks before writing them.

Of course, Shielder participants do occasionally break promises, but not usually intentionally. If they intentionally break a promise, they must pay the consequences dictated by the rules as in Step 7 as shown in FIG. 6 and also risk putting their abilities to form Mental Shields in total jeopardy, as described in the “Locking Mechanism” as shown in FIG. 5B, which is the last thing most Shielder participants want to do. Most Shielder participants come to value the benefits so much that they don't want to be without them even for 24 hours, the rule penalty for breaking a promise, the Step 3 and the Step 7, as shown in FIG. 6. It should be noted that the “locking mechanism” may be taught any time before or after this point.
Step 7: Teach and help Shielder participants learn the techniques and tips. The Shielder participants are taught with techniques developed to use with this promises-tracking method of the present invention, while continuing to monitor their progress and give support. These techniques are found to be very effective in helping the Shielder participants stay in control even when their promises are over. The techniques are numerous and, in the preferred embodiment, are taught during a series of multiple individual lessons as the Shielder participants continue making promises and gaining skill. Some or all of these techniques may be taught before or after this point, as desired. According to the preferred embodiment, weight loss is made as an example. With or without obvious changes, these can be altered for other types of behavior goals. Other related tips and suggestions may be given, such as health tips and ways of doing things that we have found work best.

Tips and Techniques for using Mental Shields for Weight Loss: During the weeks while learning to “Shield” and practicing, the following tips and techniques are taught: (The same techniques may be used for other types of behavior with or without alterations.)

During the training period, Shielder participants may be taught a technique for learning that they don't really want to eat without restriction, as they might think they do. This “Pig Out” technique is usually used with supervision. First the Shielder participants are asked to add a promise to their promise hains to eat less of something they usually “pig out” on for a short time, such as a promise to have just one cookie for 15 minutes instead of their usual 10. This enables them to experience eating much less in a sure (Shielded) state of mind. After waiting the 15 minutes, they are allowed to overeat or “pig out” as usual. Based on the clinic experiments conducted, the Shielder participant are then asked about the experience. Most Shielder participants soon learn that they wished they had stopped sooner.

A variation of this technique is to request the Shielder participants to repeat the experience; but this time, rather than “pigging out” after their promise is over, they are requested to make a promise to have just a “little more” after their promise, for example half of another cookie for 15 minutes. This is called the “Poquito Mas” promise. By repeating this technique, most Shielder participants soon learn that they have the power to stop where they want and that they actually prefer to eat less food when they “Shield” the behavior. Most Shielder participants are thrilled that they have the control to eat less and actually prefer less.

With the “Brake Shielding” promise, a Shielder participant promises to have a limited amount of something for a short period of time. Before or after the end of the promise, he or she makes another promise to have a little more for another period of time. This may be repeated as many times as desired. Like a big, heavy truck going down a steep grade, they are “applying brakes” to avoid going out of control.

The above 3 techniques in the Step 7 are effective for “don't” behaviors, such as “don't” eat so much. These techniques can be used in reverse for “do” behaviors, such as “do” exercise:
(a) The “Delay Quantity” technique is reversed by asking the Shielder participant to promise to “do” a desirable behavior for a limited or short period of time to experience the behavior in the state of mind of being Shielded.
(b) The “Poquito Mas” promise is reversed to “do” a little bit of the desired behavior, rather than just stopping altogether.
(c) The “Brake Shielding” promise is reversed to promise to “do” a little of the desired behavior for limited or short intervals, like an overheated truck going a little way up a steep hill and then stopping to rest or cool down as needed, but able to make steady progress towards its goal.

The Shielder participants are encouraged to “set their own stopping point,” rather than continuing to their old stopping point, which, in some cases, is when they literally, physically can't eat any more (a Physical Shield).

To stimulate desires to make promises, the Shielder participants are often encouraged to pay attention to their behavior desires with the “What do you really want?” technique. For example, do they really want to eat the 18 cookies in the box they have just opened? Or would they rather protect themselves from too many with a promise?

One of the most important techniques is the “Try and Compare” technique. Shielder participants are often encouraged to try a behavior that is difficult for them by Shielding or promising to engage in the behavior for a short period of time, and to notice how much easier it is to accomplish in a “Shielded” state of mind. Then to compare their old behavior to the new experience. If they want to make the desirable behavior a habit, they are encouraged to make promises to repeat the new behavior until it becomes easy and automatic.

The Shield Method of the present invention may pattern techniques after the behaviors of successful role models in areas in which the Shielder participants wish to change or improve. For weight loss, for example, techniques may be patterned after the way “Naturally Thin Eaters” eat. These successful role models eat only when they are hungry, only until they are just full and don't usually eat between meals. Over-eaters, on the other hand, generally eat whether or not they are hungry, eat beyond being full and often eat between formal meals.

The many “positive aspects” of following the naturally thin eater model may be pointed out to the Shielder participants, such as being able to look forward to meals with a good appetite, rather than already being full; being able to eat anything they want at meals; being able to walk away from meals feeling good, rather than stuffed; being able to easily maintain a healthy weight; and etc.

The Naturally thin eaters never have to feel uncomfortable, because they get to eat when they are hungry, and it is easy for the Shielder participant to emulate. They simply estimate when they will be hungry and make a promise to wait until then. It should be noted that the positive aspects of eating this way cannot usually be achieved by over-eaters unless they are in the state of mind of being Shielded, like naturally thin eaters are.

Some Shielder participants prefer “Free Shield,” just cutting quantities and frequencies of eating as desired. Beginner Shielder participants can easily cut hundreds to thousands of calories from their eating per day, just by Free Shielding. Every time they cut 3,500 calories, they can lose another pound, since a pound contains approximately 3,500 calories, although there are other important metabolic factors to consider.

The Shielder participants are often taught to use the “Loving Parent” techniques, wherein they are encouraged to act as their own loving parent who controls what and when they eat. Parents want their children to enjoy food, but don't want them to eat too much or think about food all the time. Shielder participants are encouraged to make promises as responsibly as if they were their own guardians.

Similarly, Shielder participants may be taught techniques for Shielding their children by imposing sure parental limits. Children who only eat what and when a responsible parent feeds them are typically less obsessed with food. They can get their minds off of food for hours at a time and enjoy meals more than adults. The wrong thing to do is to let children decide what and how much to eat and allow them to become obsessed over foods. Shielder parents may also be taught how to give their children the most valuable possessions of all—positive permanent Mental Shields.

“Family Rule” and “Buddy Plan” techniques may be taught. If family members, friends, co-workers, and etc. can agree on a behavior plan, understanding and agreeing to the Shield Method concept that the Shielder participants cannot intend to break the behavior, this can Mentally Shield them. However, they should formulate rules for innocently broken promises to avoid unnecessarily breaking their Shields. An additional rule for this application may include having a meeting if a rule is ever broken to correct problems or impose new voluntary penalties that are sufficient to maintain the state of mind required to activate Shields.

The “How much can you cut for 5 minutes?” technique. The clinical test results find that it is almost always better (and more healthy) to decrease the term of a promise rather than to increase the quantity of eating (or quantity of other undesirable behavior). For example, it's better to promise “No more than one cookie for 5 minutes” rather than “A whole box of cookies and nothing else for 2 days.” But the id portion of the mind will resist making restriction promises. Therefore, the Shielder participants are encouraged to mentally consider how much they can cut for just 5 minutes. If they feel they can cut down to 1 cookie, the Shielder participants are requested to consider increasing the amount of time of the promise. Can they promise just 1 cookie for 15 minutes? How about just 1 cookie for 30 minutes. Whatever amount of time they feel comfortable with; the Shielder participants are encouraged to Shield or promise. In fact, it is best to Shield the first easy promise they can think of and then try to improve it, to prevent changing their mind. As they soon learn, promising 1 cookie for 5 minutes “fools” their mind so they can easily remain in control. Most Shielder participants quickly learn to stay in control and lose their “fear of food” with this (and other) techniques.

The above promise can be used in reverse for “do” behaviors, by using the “Do just 5 minutes” technique. For example, it is often too difficult for the beginning Shielder participant to promise to do an hour of exercise, but it is easy for almost anyone to promise to do just 5 minutes. We have found that getting started is half the battle. If Shielder participants promise just 5 minutes of a desirable “do” activity every day, they often get involved and do much more, especially with support. If 5 minutes is easy, the Shielder participants are encouraged to consider whether or not they can promise to do 15 or 30. Or, before or while they are doing their exercise, they can “lock in” a promise to do 10 more minutes at any moment of strength.

A related technique is the “Jack-up” or “Ratchet up” promise. While keeping a promise, a Shielder participant may have a moment of strength that he or she can take advantage of to improve or “Jack up” their promise. For example, while keeping a promise to have two cookies, the Shielder participant may have a moment of strength to promise not to have the second cookie. Just being able to control their eating behaviors makes the Shielder participants feel stronger, so it is not unusual for them to have moments of strength to improve their promises.

The id portion of the mind will resist long-term promises, since it desires to keep pleasure possibilities open. However, it is much easier for the Shielder participants to promise not to eat for 5 or 6 hours if they can promise to have a little snack during the 5 or 6 hours in case they want it or get too hungry. With the “Security Blanket” promise, the Shielder participant simply makes a promise like, “No eating for 5 hours, except I can have a cup of yogurt if I want it.” This makes it easier to make longer-term promises.

The “Productivity” promise. The Shielder is encouraged to Shield or promise activities other than eating. Shielder participants are often encouraged to take advantage of “Automatic Shields” for desirable behavior whenever possible. For example, a person who lives in a house in which they have to climb 2 dozen steps to get to their front door is “Shielded” to do stair-stepping, without having to make promises. Since the person feels “absolutely sure” that he or she has to climb the stairs, his or her mind helps him or her or her accomplish the exercise easily and comfortably, without even thinking of it as a chore. Other Automatic Shields include not buying a car, so you have to walk everywhere, and having a dog who “expects” to be walked every day.

Just having the ability to Shield “at will” can “reverse the activity of the mind.” Whereas before, an over-eater's mind would always “flirt” with ideas of engaging in worse behavior, now their minds are always “flirting” with ideas of making promises to engage in better behaviors. The Shielder participants are encouraged to enhance this reversed state of mind by using a technique of “Flirting” with ideas, plans, visualizations and rationalizations of how they could do better, not worse.

The Shielder participants may be taught the “Stop the Hand-To-Mouth Reflex” technique. Interrupting the “hand-to-mouth” reflex of eating, even for a few minutes, seems to decrease urges to eat more by signaling the brain to stop eating.

One of the most effective and easy Shielders techniques is called the “Promise Too Much,” technique. The Shielder participants promise not to have any more than they think is “too much.” For example, when a pizza arrives, the Shielder participants may think they “should” promise to only have 2 pieces, but their id makes them hesitate. Yet they think that 4 pieces would be way too much to have, and promising to have no more than 4 pieces would be an easy promise for them to make. The Shielder participants are encouraged to make this easy promise immediately, to avoid eating even more than 4 pieces feeling so bad about their behavior that they just keep eating. This technique prevents the Shielder participants from eating even more than they think is too much, and can be used as leverage to improve the promise at a moment of strength. It is found that once they make the promise to have no more than the amount they think is “too much,” the fact that the “danger” has been eliminated makes them feel so relaxed and confident that they can easily improve the promise. This is also called the “Eliminate the Danger” technique.

There is a technique that Shielder participants can use to gently reduce their appetites called the “Eat Down Your Appetite” technique, wherein the Shielder participants have what they want but less. When promised consistently, this can reduce or eliminate low blood sugar or diabetes by actually decreasing the size of the Islets of Langerhans of the pancreas which produce insulin, according to the Guyton's Textbook of Medical Physiology. The person may no longer suffer symptoms of light-headedness, shakiness, fatigue or extreme hunger when they eat less, since their insulin response to eating will not overly deplete the blood of sugar.

The Shielder participants are often encouraged to use the “Get Satisfied” promise. They learn that they can only truly get satisfied when they Shield a limited amount of food. Otherwise, their mind will keep working for more and more food. Over eaters learn why they may have been eating without ever really getting satisfied and learn to enjoy “less food” more than “more.”

The “Have It Later” technique taught the Shielder participants that they don't have to have things they want immediately, and they don't have to deprive themselves of things they see and want when Shielded. They can buy or take things to enjoy later.

The Shielder participants are often taught to take advantage of the protection that other kinds of Shields may give them. For example, the support group meeting utilizes strong “Social” and “Competition” Shields to help motivate the Shielder participants. Such support group meeting can be arranged by and held through the promises-tracking device 1 for the Shielder participants. Not bringing especially tempting foods home can “physically” Shield a person while at home. However, most experienced Shielder participants can easily control their behaviors at home, even with the strongest temptations in their kitchens.

The Shielder participants are often taught the “Out of Sight, Out of Mind” promise. Putting away the food they have Shielded not to eat is a good policy to reduce forgetting that they promised not to have more and reduce temptations.

With the “Bite the Bullet” or “Just Make It” technique, the Shielder participants are often encouraged to “just make” some promises that are difficult for them at a moment of strength for the purpose of experiencing new behaviors in the state of mind of being Shielded.

The Shielder participants are often taught to “Shield their intentions” and “Shield their plans.”

The “Predict the Future” technique, is thinking ahead and making promises to prevent undesirable behaviors. When a Shielder participant thinks ahead, he or she can make promises to avoid behavior disasters.

One favorite technique is the “3 more bites” promise because it works almost like magic during the clinical experiences. When a Shielder participant finds himself eating, such as from a box of crackers, and can't seem to stop, the Shielder participant is taught to ask himself or herself how many more crackers or “bites” he or she will allow himself or herself to have and Shields it. This makes it easy for him or her or her to stop and helps the dragons of his or her mind prepare him or her or her to feel satisfied at the exact point he or she is Shielded to stop. Beginner Shielder participants usually can't promise to have just “3 more bites,” but they are encouraged to think of the lowest number of additional bites that they can keep comfortably promise. Even 10 more crackers are better than the whole box. Experienced Shielder participants can usually easily promise just 1 to 3 more.

There is one condition that the Shielder participant may actually be encouraged to break promises and pay the consequences. That is when they are having trouble committing to keep their promises. When a Shielder participant has trouble committing to keep his or her promises, it usually means that the consequences of breaking promises are not strong enough to Shield them. The Shielder participants should not try to force themselves to keep their promises with “willpower”.

Instead, such Shielder participant should be encouraged to use the “Love It Or Lose It” technique, in which the Shielder participant is actually encouraged to break one or more promises and pay the consequences, so that he or she can learn how important his or her ability to Shield really is to him or her or her. This is sort of like asking a person to stop paying his or her bills so that he or she can learn how important his or her credit is to him or her or her. Once the Shielder participant learns that he or she can't have reliable personal control if he or she is not reliable about keeping his or her promises, he or she usually has no problem committing to keeping his or her promises.

A technique called “Stop the Mind Battles” is taught to help decrease or eliminate conflicts between what a Shielder participant feels he or she “should” do and what he or she “wants” to do. By making a promise, the behavior decision is made and this stops the mind battle over what to do. Even making a short-term promise can alleviate the discomfort of a mind battle. Mind battles only happen when a person is not “Shielded.”

One of the most important techniques is the technique to “Establish New Habits.” The steps for establishing a new habit are to decide on a habit goal include:
shield to “try” the behavior in the state of mind of being Shielded (with the mind working for the behavior instead of working against it);
compare the new behavior experience to the old behavior; and
shield the desirable behavior regularly, with support until it becomes easy, automatic and preferred.

A final (optional) stage “proves” that the habit is firmly established, if the person is actually revolted by his or her old behavior or would never go back to it.

When teaching the Shielder participants how to “Shield,” a technique of telling the Shielder participants a fictional story about “The Dragons of the Mind” may be used, who form the most powerful team known to man for obtaining desires. In a symbolic way, the following story clearly describes the nearly infinite power of the mind and why it is almost impossible for a person to succeed at a behavior goal when he or she has to try to fight the dragons of his or her mind. It explains what is required to activate Mental Shields and how these Shields can put the power of the mind to work “for” a person's plans, rather than “against” them. The main characters of this story are id the dragon, who represents the primitive mind and commands a host of Dragon workers, Judge Ego, the Shield Master who rules for or against desires and the Superego Jury. Humanoids sit in the Superego Jury box who look like the Shielder participant's parents, church officials and people they look up to. The Superego Jury makes the Shielder participant feel good when he or she does what he or she feels he or she should and makes him or her or her feel bad when he or she doesn't. Id the dragon is primitive in that he works for an individual's current desires without distinguishing between whether or not they are good or bad for him. Id can put the full power of the mind to work for desires, even when they are not very strong, and even cause desires to be intensified. For the first time, the Shield Method gives the Shielder participant a way to control or “Shield” Id for personal applications and get his or her mind working for positive desires and goals.

There are a number of “Fun Shielding Techniques”, such as the “Mini Smorgasbord” technique, in which the Shielder participant has a small amount of all the foods he or she wants. For example, he can have ⅙th of 6 donuts (instead of 6 donuts) for a variety. It should be noted that only the Shielded participants have the control for this to be fun, that is only when they are in the “Shielded” state of mind of being “absolutely sure” that they won't have any more than they plan.

The Shielder participants often automatically make their meals “special” in some way, such as set a place setting, use special plates, and etc., even though we don't require them to. This is difficult for the Shielder participants who have not gone through the program to understand, but can be easily explained by the following example:

When a Shielder participant “Shields” to have only ½ cup of ice cream, so that he or she feels “absolutely sure” that he or she is not going to have even one more bite for a few hours, he or she automatically finds himself or herself carefully measuring the ice cream, putting it in a tulip glass with a fancy long spoon, setting it on a doily and planning to have it right when his or her favorite TV show starts. Yes, he or she does lick the tulip glass, but he or she doesn't have even 1 more bite than he or she Shield.

It is noted that years ago, scientists noticed that naturally thin eaters do things like this. They often set place settings before eating, wait until they sit down before starting to eat, arrange their food pleasantly, etc. The scientists theorized that if they could “train” over-eaters to imitate such behavior, they could change their habits. This “behavior modification” training does not work for most people, otherwise it would certainly be the treatment of choice for obesity. What the scientists didn't realize is that it's difficult and even mentally painful for people to try to eat like this unless they are “Shielded,” like thin eaters are. Then the behavior is often an easy, natural result, as in the ice cream example, above.

The Shielder participants may be taught the “Not hungry? Shield time!” technique, in which the Shielder participants ask themselves at any time “Am I hungry?” If not, they are encouraged to Shield not to eat for any amount of time that they can.

The “You Can Take It With You” technique is another effective and fun technique, in which the Shielder participants learn that they don't have to feel deprived. When they are Shielded and see a food they really want, like a pastry, they can take it and have it after their promise is over.

With the “Diluted drinking” technique, the Shielder participant learns to dilute caffeine and alcohol drinks to the extreme, turning 1 iced tea into 6, or 1 6 ounce glass of wine into 6 wine coolers. The Shield Method of the invention helps the Shielder participants control alcohol consumption just as it helps them control their eating.

The Shielder participants are encouraged to learn a technique called “Regulate Your Hunger.” It's always easy to estimate when they will be hungry after a promise is over and they can make a promise to wait until then. The Shielder participants have the control to have a small snack to “tide them over” so that they can be hungry at any chosen time.

Another fun technique or game, called “Spot The Shield” can be used to find out what Shields a Shielder participant has. The Shielder participant can be told if a person has a Shield for a particular behavior by his or her habits and how his or her mind is working. For example, if a person is asked if he or she ever eats when he or she is not hungry and he or she responds “No” or “Not usually, the person is most likely a naturally thin eater and has a permanent ”Healthy Eating Shield.“ People who don't fight over food like wild animals in social situations have healthy ”Permanent Social Shields“ for this situation. A person who scowls when the idea of abusing drugs is brought up most likely has a healthy, desirable Permanent Shield against drug abuse. If a person isn't listening to reason, he or she is probably ”Shielded for“ a certain desire that his or her mind is suppressing the negative aspects and exaggerating the positive aspects of having. These “Spot The Shield” techniques may have important applications in fields such as criminal science and child rearing.

It is appreciated that the above techniques can be programmed in the program operated electronic device 1 to works as the promises-tracking device of the invention, so as to teach and guide the Shielder participant during the processing of the steps (a) to (g) of the promises-tracking method by describing and illustrating on the screen 13 at the time such as the promises-tracking device determines the Shielder participant is breaking a promise, when the excused broken promise button 113 or the unexcused broken promise button 114 is activated, when the activation signal is generated through the activator 112, the timer setter 111 is setting, under inquiry by the Shielder participant, and etc. The promises-tracking device 1 can be programmed with artificial intelligence software or supported and linked to an artificial intelligence online platform to determine the appropriate technique(s) to be taught according to the records of the added promises in the promise chain and the broken promise records of the Shielder participant so as to guide and encourage the Shielder participant to keep the promises in the promise chain.

Many of the above techniques can be used extensively for other behavior applications, such as legal, financial, social etc. For example, when you write a contract to buy on an installment plan, you are using the “Brake Shielding” technique for “do” behaviors. The present invention is the first to use these techniques effectively and practically for personal control applications.

Many of these techniques have also been used for weight loss before, such as the “Predict the Future” technique of thinking ahead and taking action to prevent undesirable behaviors. But there has been no reliable way to ensure success in following them. Now, when a Shielder participant Shields this behavior, he or she is practically guaranteed to follow it. Contracts (another form of promises) have been tried for weight control, but have had very low rates of success and it is not possible to make or upgrade contracts instantly, any time and any place, as Shielder participants can do.

Therefore, the Shield Method is the “missing ingredient” needed to make all of these (and many other) techniques effective. Trying to use these techniques without utilizing Mental Shields has been like it would be if we had to try to pay our bills if we didn't feel “absolutely sure” that we had to.
Step 8: Provide ongoing support and new applications. As the Shielder participants continue with the promises-tracking method of the invention, the promises-tracking device 1 continue to monitor the Shielder participants and provide support and motivation. The primary objectives are to encourage the Shielder participants to set and work on goals, prevent them from forgetting what they have learned and help them use the Shield Method consistently to the point of reaching their goals and changing their habits. They may reach a point that they no longer need to “Shield” behaviors because they prefer their new habits.

In preliminary tests, it is noticed that when Shielder participants receive adequate support, such as weekly appointments or meeting, most stop being out of control, lose their fear of food, have increased self-esteem, change their behaviors dramatically and quickly start to develop new habits that they prefer. Weight loss is a side effect of the control.

Personal support, in the office or clinic with the support of the promises-tracking device 1 or through internet via the promises-tracking device 1, seems to give the best results. With personal counseling or support group, it is not unusual for the Shielder participants to be able to decrease their food intake by ⅓ or ½ with little effort. Support can be essential because the id portion of the mind may soon begin to resist the idea of making promises. With support, it is noticed that the Shielder participants continue making promises consistently until their new behavior becomes easy, automatic and preferred, at which time they may no longer need to make promises for certain behaviors. Inexpensive support group meetings are highly effective, but any other types of support, such as personal, non-personal, interactive computer or device, and etc., that can help the Shielder participants to set goals, continue making promises, increase their skill and prevent them from forgetting what they have learned will increase their ability to succeed.

At the end of the teaching process, the Shielder participants often can't believe how much they've changed in such a short time, and so easily and gently. Most can do better than before in almost any situation and can rapidly regain control any time they start going out of control. It is noticed that after a while, the number of strong urges to binge or overeat may decrease considerably or almost completely stop. This is due to the establishment of a new, permanent “Mental Control Shield.” The person feels “absolutely sure” that he or she can maintain control, so the ego portion of the mind forms a protective Mental Shield against many id desires, so that when the id sends a desire into the consciousness, the ego can deflect it. The id is persistent, but not foolish. It won't work for “impossible” desires, so it eventually stops sending so many desires.

New habit development is the greatest achievement of the Shield Method. The Shield Method provides the promises-tracking method that really can change habits, dramatically, very quickly and permanently. No other conventional method has been able to so easily, quickly and effectively promote new habit development. Most doctors and scientists now agree that establishing improved behavior habits is the only sure way for people to be able to lose weight and maintain their weight loss long-term. The fact that habits are preferred is a great bonus of the Shield Method, since preferred habits are more likely to last a lifetime.
Step 9: Unique promotional themes, slogans, floor plan, etc. Unique advertising and promotion are geared to orient and educate potential Shielder participants about the benefits of the Shield Method and to interest them in programs that may benefit them. A unique advertising and decoration theme on the screen 13 of the promises-tracking device 1 based on Shields and dragons, medieval or other, may be used. The dragon theme originates from a story that is told during the teaching process of the Shield Method, about imaginary dragons that live in the mind, to more easily teach complex concepts of how the mind works. Characters and elements of the story can be utilized in advertising and in other ways to develop strong associations, in the public mind, with the Shield Method. Further, due to the fact that the Shield Method gives most people much more “willpower” than they have ever had, the term “Super Willpower” as a slogan can used on the screen 13 of the promises-tracking device 1. A unique theme arrangement of the Shielder participants and instructor attending a group support meeting online through the promises-tracking device can be designed to enhance the meeting effect and efficiency. Information in the mind of the Shielder participant can be helped through “chanting answers”. For example, at a meeting the instructor may call out or chant like a drill sergeant, “What happens when you don't Shield to do something you don't have to do? The Shielder participant may then reply in unison, “It isn't likely to get done!” These may be musical chants and may be used in exercise meetings.

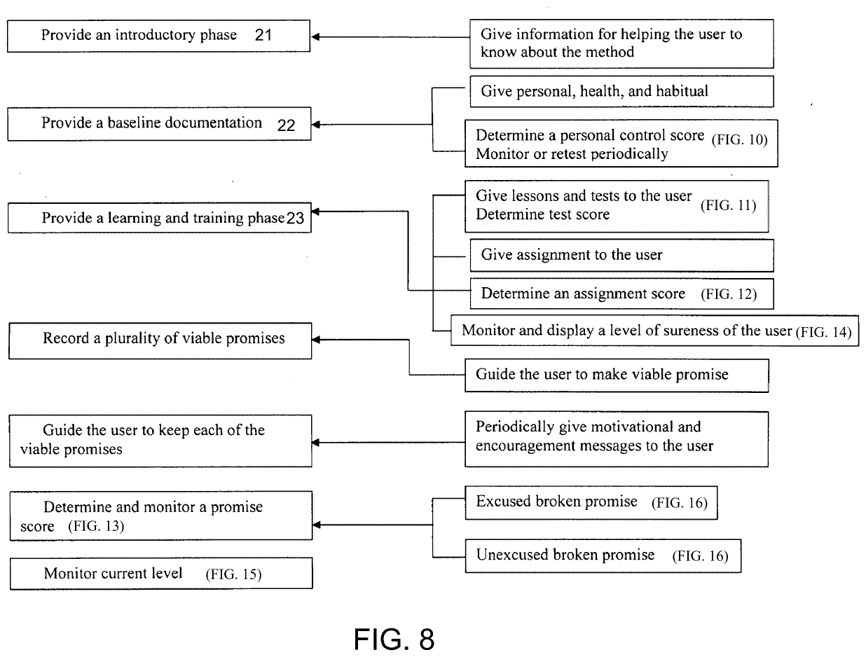

Referring to FIG. 8, a plurality of different phases for the participant is illustrated, including an introductory phase 21, a baseline documentation phase 22, and a learning and training phase 23 to the participant, preferably provided before the step (a), and/or during the step (a) to step (g).

The introductory phase 21 is arranged for introducing the promises-tracking method of the present invention to the participant. The baseline documentation phase 22 is arranged for evaluating what kind of lesson and assignment are given in learning and training phase. The learning and training phase 23 is arranged for learning and training the promise techniques as described above and shown in FIG. 6. After receiving the learning and training phase 23, the participant becomes a Shielder participant of the promises-tracking device 1 and the method thereof.

In the step (a), the plurality of viable promises made by the Shielder participant are recorded in a chainlike manner to form the promise chain. The Shielder participant is guided to make the promises after the learning and training phase 23.

The step (b) and step (d) are guiding steps for the Shielder participant to guide the Shielder participant to keep each of the viable promises from the promise chain, wherein the Shielder participant is enable to make the viable promise to endure situations that the Shielder participant finds difficult, so as to guide for helping the Shielder participant endure the difficult to keep the viable promises instead of fighting his or her efforts to do so.

In the step (e), the promise score of the Shielder participant is determined and monitored regarding to the promise chain, which is an incentive to motivate the Shielder participant to keep the promise score as high as possible, wherein when one of the viable promises is broken, the promise chain is then broken to have a negative effect on the promise score.

According to the preferred embodiment, the promises-tracking device is preferably programed from the program operated electronic device 1 or a program installed into the electronic device or being accessed through Internet. The device can be a hand held electronic device with the screen 13, such as a touch screen, wherein the program is pre-loaded in the hand held electronic device. As described above and shown in FIG. 3, the program operated electronic device comprises the at least one memory modules 12 and the processor 10. The information of the introductory phase 21, the baseline documentation phase 22 and the learning and training phase 23 can be stored in different sections of the at least one memory module 12.

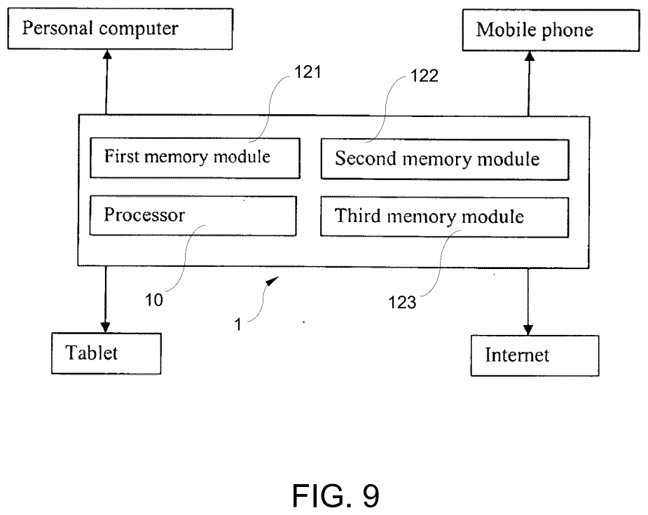

Alternatively, as shown in FIG. 9, the promises-tracking device 1 can be an independent program operated electronic device 1′, which comprises the processor 10 and three memory modules 121, 122, 123, communicatively links with a personal computer, a mobile phone, a tablet, and/or an internet platform. The first memory modules 121 is configured to stores different information of the introductory phase 21, the baseline documentation phase 22, and the learning and training phase 23, wherein the different phases 21, 22, 23 are arranged to be displayed on a screen 13 provided on the program operated electronic device 1′ or the screen of the personal computer, the mobile phone or the tablet.

The second memory module 122 is configured to store the viable promises in the promise chain of the Shielder participant. The third memory module 123 is configured to store guidance information for guiding the Shielder participant to keep each of the viable promises from the promise chain, wherein the guidance information is arranged for enabling the Shielder participant to make the viable promise to endure situations that the Shielder participant finds difficult.

The processor 10 is arranged for determining the assignment score and the promise score. Accordingly, the Shielder participant is able to selectively input the broken promise as one of the excused broken promise and the unexcused broken promise via the input device 11 of the program operated electronic device 1, such as a keyboard or a touch screen.

The program application programmed in the program operated electronic device 1 to form the promises-tracking device is designed to help the participant who is interested in behavior control such as for weight control, to stop smoking, for control of alcohol, for drug abuse, and etc. As shown in FIG. 6, to qualify prospective clients and record before status, information for determining whether the promises-tracking method can help the client is provided, which clearly describes and states which individuals the promises-tracking method is likely to help. Since the purpose of the promises-tracking method of the invention is to increase self-control, if a person's problem is due to other cause(s), the promises-tracking method may not help him. The participant may select to download the appropriate program due to his or her need of enhancement of behavior control to his or her program operated electronic device 1.

For example, foe the “eating control” App, this includes individuals who lack control of their eating behaviors which results in “over-eating” (eating too much and/or too often). They may or may not be overweight. If they are overweight but not from over-eating, they may be told that this program is not likely to help them and be advised to consult a doctor.

After the appropriate program is downloaded to the program operated electronic device 1, the program then enters the INTRODUCTORY and ORIENTATION PHASE to orient and motivate new participant, as shown in FIG. 6, in which information is provided about the purpose of the program, its benefits and an overview of how it works, as described above. For example:

“This App will enable you to master the “Shield Method” App for weight loss”;

“The Shield Method is the first method that can give people SUPER WILLPOWER for personal control. If you are an over-eater (you eat too much and/or too often), this program can give you SUPER WILLPOWER in eating situations, so you can easily slash hundreds to thousands of calories from your daily eating—without feeling deprived in the least. For the first time, weight loss can be easy and stress-free!”

“Yet the Shield Method is more like a game than a cold, clinical method. You will simply establish a mental chain of easy promises that you won't break, “no matter what.”* Then, every time you add a promise to your chain, you will lock-in better behavior. (*Note: whenever “no matter what” is used in this context, it means “except when excused by the rules,” which are included in the steps below.)”

“But this powerful method is much more than just a game. It actually programs your mind to help you keep your promises! This is because it enables you to cross the threshold of SURENESS, which is required to program your mind. Once you are SURE that you will keep your promises, your mind will be programmed to help you keep them, resulting in super easy and super exhilarating personal control—your mind will be doing all the work! This is the same SURENESS that most people experience when they start a diet. They can sail by the donuts and other illegal foods with ease, because their mind is protecting or “Shielding” them from feelings of discomfort! But their SURENESS just doesn't last. With the Shield Method, it does!”

The App will then give an OVERVIEW of what the promises-tracking method will do for the participant, such as it will help the participant to use the device and establish his or her promise chain.

Then, the App will teach the rules and tips to the participant, such as how to make amazingly clever promises (called the “Promise Techniques”) that will enable him or her to eat significantly less of regular foods—not diet foods—without even missing what he or she is cutting out. It will guide and motivate the participant to succeed. Additional benefits of the app may be described. These include increased physical and mental health; reduction of stress and “mind battles” (battles in his mind over whether or not to do what he feels he “should” do); reduction of stimulations from the mind to do what he feels he “shouldn't” do; reduced feelings of restriction, deprivation, fear, guilt, and easy, comfortable, reliable personal control to achieve other kinds of personal goals., such as chores or exercise.

Referring to FIG. 8, the promises-tracking device 1 will then enter the BASELINE DOCUMENTATION PHASE 21 in which “before” statuses are documented, prior to starting the learning phase of the program, to get baseline scores that can be improved upon:
(1) The participant will be prompted to enter his or her CURRENT STATUSES into the program, such as:
(i) His or her bodily statuses, such as height, weight and measurements.
(ii) His or her level of personal control in areas related to his goals (his ability to stop eating after having sensible portions, control quantities, etc.).
(iii) Other statuses related to his problem and/or goals (such as how his or her weight problem affects his or her emotional state or prevents him or her or her from participating in certain activities he or she is like to do).
(iv) The program will obtain these statuses by asking a series of questions pertaining to his current levels, in order to get baseline scores that can be improved upon. The program will group these by type of behavior, such as: “willpower”; “ability to stop eating”; “food choices”; “problems with quantity”: etc. The participant will select one of 9 options to describe his current status level: −4 (Never); −3 (Almost Never); −2 (Very Rarely); −1 (Rarely); 0 (Moderately); +1 (Often); +2 (Very Often); +3 (Almost Always); +4 (Always). An example of a question is:

“How often can you set a sensible point to stop eating and feel satisfied and ready to stop at that exact point?”

The program will ask the participant to think carefully and select only one answer; truthfully, to the best of his knowledge. For example, if he selects −3, it means that his current level of control with his or her behavior is quite low.

If he or she accidentally selects more than one answer, the program will ask him or her to select just one. If he seems to be having trouble, there may be a “help” function or suggestions pop up on the screen 13.
(2) After answering all the questions, the program will compute the participant's “starting” STATUSES for all categories and store them in a chart as “Beginning Scores.”

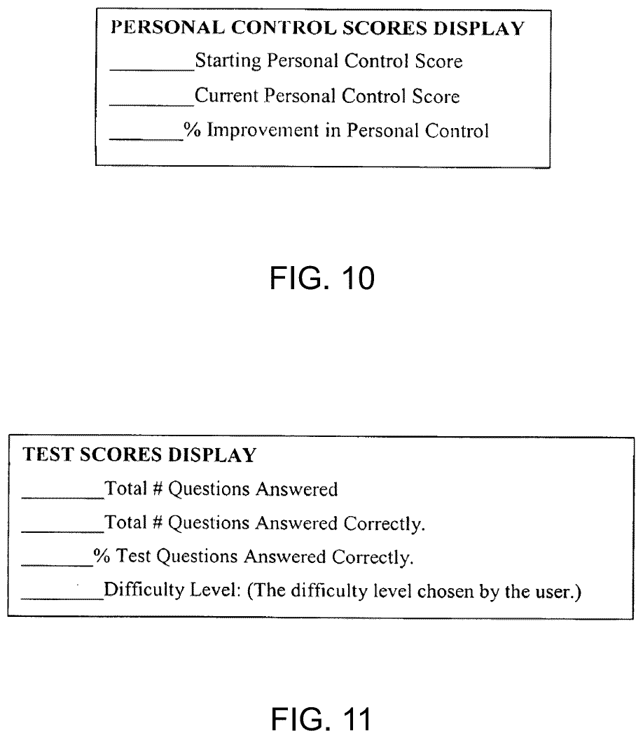

This chart will provide at least 3 comparison scores: starting score, current score, and overall improvement (the “current” and “overall” scores to be filled in later). Every retest score may be listed for comparison. Some or all scores of these scores may be displayed on the face of his device so that he can easily see them. For example, a PERSONAL CONTROL SCORE of the participant is shown in FIG. 10.

The participant may have the option of displaying some or all of his or her scores on the face of other devices, such as his iPhone and/or iBook; or on a social-network program such as “Facebook” to announce to others how much or little control he started with and how these change as he advances through the program, for motivation to do well.
(3) The participant may be prompted to state the “primary problem(s)” that he or she wants to the program to help him or her solve (such as being overweight).

The participant may be prompted to give a “case history” of this problem(s):
(i) How long he has had this problem (how long he has been overweight).
(ii) Other methods or treatments he has tried: when, for how long and the results (for example, the participant tried a low carb diet for 6 months, lost 15 pounds but regained it over 6 months).
(4) The participant may be prompted to fill out a “symptom survey” of additional related problems that the program may be able to help him or her with (such as health problems, stress and self-esteem problems).
(5) The participant may be prompted to list or select goal(s) (such as to lose a certain amount of weight, permanent weight loss, etc.). For example, the participant may be asked to list or select reasons for why he wants to obtain these goal(s), (such as for improved appearance and health reasons).
(6) The participant may then be prompted to enter personal “Before” information (starting information about his health conditions, medications he may be taking, etc.).
(7) His or her current eating patterns may be documented: when he or she eats, how much, types of foods, etc.

Referring to FIG. 8, the program will then enter the LEARNING AND TRAINING PHASE 23, which will teach the individual the Shield Method, using lessons, assignments and tests.

Lessons, which will be given by written format with illustrations and/or animations or other format, teach how to use the promises-tracking device 1 and how to establish and use the chain of promises through the promises-tracking method of the invention.

Each lesson will usually be followed by a test. Optionally, the lessons may be interactive, so that test questions are given as he takes them. The participant will be informed that it is important to learn the lesson materials thoroughly. To help motivate the participant to do so, he or she is told that his TEST SCORES will be displayed. These scores will be recorded by the promises-tracking device 1 on a chart and/or displayed as: total number of questions answered; total number of questions answered correctly; total number of questions he or she answered incorrectly; overall percentage of correct answers, as shown in FIG. 11.

The participant may be told that his or her test scores are unalterable and permanent, to help motivate him or her or her to get the lowest number of incorrect answers and the highest overall score. The participant may have the option of displaying his or her test scores or his or her overall test score on the face of his or her iPhone, on his or her iBook, or on a social-interactive program such as “Facebook,” etc. to show others how well he or she is doing on the tests, to further motivate him or her or her.

If it is determined from the participant's test answers that he or she has a serious misunderstanding that could impede his ability to improve one or more levels of control, he or she may not pass the test, then:
(i) The program may prompt him or her or her to retake the lesson, review information or obtain help from a trouble shooter, online support group, counselor, and etc.
(ii) The participant may not be allowed to proceed to the next lesson if he or she does not pass a test.

If it is determined from his or her answers that he or she has passed the test, then he or she will be allowed to proceed with the program. The program will add his or her test scores to the chart and display(s) as illustrated in FIG. 11.

Referring to FIG. 8, proceeding with the program, assignments may be given between lessons, such as to make promises and practice techniques. The program may require the participant to take a certain period of time for assignments. A “Pre-test” may be given before his or her next lesson to determine if he or she completed the assignment and had the desired results.

If it is determined from his or her answers that he did not do the pre-test; or did have the correct answers or satisfactory result, he or she may not pass the pre-test. Then, the importance of doing the assignment correctly will be re-stressed. The participant may be prompted to learn information more thoroughly by retaking one or more lessons or a part of a lesson; or to obtain help, such as from a trouble shooter within the program, online support group or counselor.

The participant may be required to repeat the assignment until he or she gets the desired result, before being allowed to continue with the program.

If it is determined from his answers that he or she passed the pre-test (did the assignment and/or obtained the desired result), then the participant will be allowed to proceed to the next lesson. The program may add the pretest scores to the test scores as shown in FIG. 11 and will compute his or her assignment score and record it.

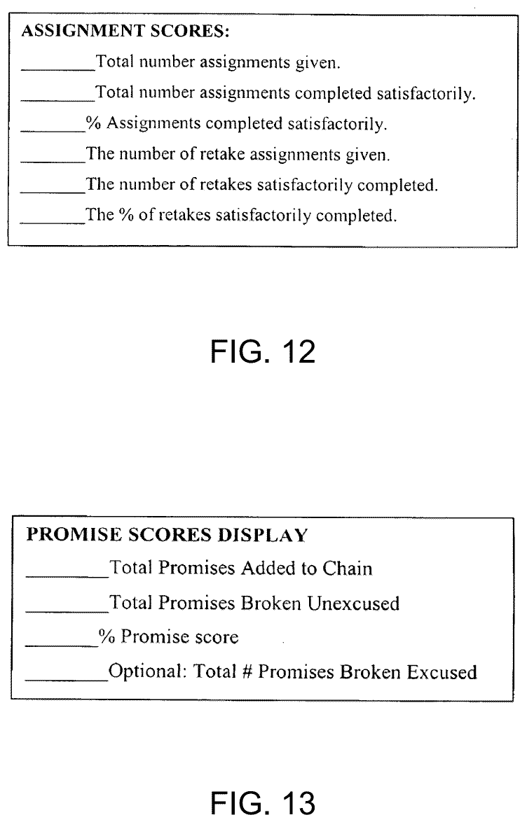

As shown in FIG. 12, an “Assignment Score” may be displayed, wherein the total number of assignments given; the total number of assignments satisfactorily completed; the % of retakes satisfactorily completed; the number of retake assignments given; the number of retakes satisfactorily completed; and the % of retakes satisfactorily completed.

The participant will continue taking lessons and tests and doing assignments until he or she has completed them all. The participant will be able to make promises while taking the lessons and afterwards, indefinitely. As shown in FIG. 13, a “Promise Score” will be calculated and displayed.

Periodically, while learning the program and continuing indefinitely afterwards, the participant will be prompted to re-enter his statuses to document changes in his Personal Control Score (as shown in FIG. 10). After re-answering the questions, the software will re-compute the totals and enter his or her new current status scores in the Personal Control Score Display and in the charts. The program will compute and display the % improvement in the report and displays.

The participant will be able to re-read lessons and re-take tests: the program may add the resulting tests scores to the Test Score Display (as shown in FIG. 11).

The individual may be able to re-do assignments: the program may add the resulting assignment scores to the Assignment Score Display (as shown in FIG. 12).

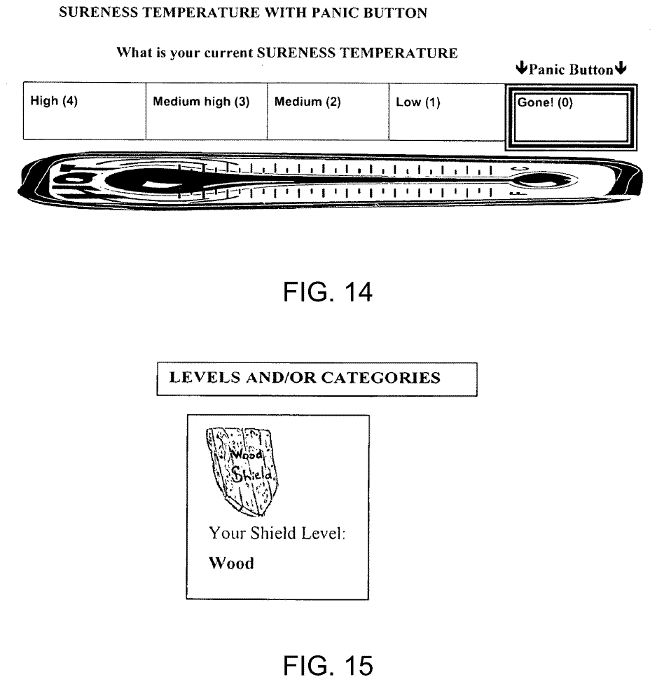

Referring to FIG. 14, a “Panic Button” may be available on the screen 13 for the participant to press or access if he or she loses confidence in his personal control, such as if he or she is in a stressful situation. The participant may simply press the panic button that will result in lesson material, tips, suggestions and/or techniques as described above to be provided to help restore his or her situations and confidence. The participant may be prompted to record or log the situation, the result and what he or she learned from it.

Further, a “Sureness Temperature Display” may be available to monitor the participant's level of “sureness” that he or she will keep his or her promises (and/or eat half each time he or she eats). This may be steady on the screen or may appear daily, periodically or whenever there are indications that the participant's level of sureness is lowered. The participant may be prompted to enter or tap his current level of sureness when prompted to, such as when the temperature display comes up on the screen.

If his or her level of sureness drops, lesson material, tips, logs, suggestions and/or techniques may be provided to help restore his or her sureness. The participant will be given an option to stop the session and finish answering the questions later.

If the participant is interrupted from lessons, tests, etc., the program may remember where he or she left off when he or she restarts. Stories, examples and analogies may be used, throughout the program, to help him or her or her understand the concepts being taught.

Referring to FIG. 6, the participant is helped to establish his or her promise chain and teaches effective Promise Techniques as described above for adding promises to his or her promise chain for easy, comfortable, lasting personal control—using lessons, illustrations (which may be animated), assignments and testing.

Firstly, the participant is informed of the purpose and benefits of the promises-tracking method. Information about the difference between following a behavior plan when his or her mind is FIGHTING the plan and when the mind is programmed to HELP him or her or her follow it.
(i) The participant is taught and given examples that demonstrate that if he or she has to FIGHT his or her mind to follow behaviors that his or her primitive mind would fight, it can be difficult and uncomfortable to try to follow them; and he or she will have unreliable success rates for doing so. For example:
(ii) The participant is taught about the mind's powerful “mind-tools,” as described above, which can make it difficult and uncomfortable to follow his plans when he or she has to FIGHT his mind to try to follow them.
(iii) The participant is warned that, for the over-eater, trying to eat less may feel like “unbearable deprivation” unless his or her mind is programmed to help him or her or she do so.
(iv) The participant is taught and given examples that demonstrate that if his or her mind is programmed to HELP him or her follow behavior plans that his or her primitive mind would fight, it's easier and more comfortable to follow them; and he or she is more likely to succeed at doing so.
(v) The participant is taught that his or her mind will use its powerful “mind-tools” to help him or her or her follow behaviors when it is programmed to help him or her or her follow them.

The concept of the “chain of promises,” a central concept of the Shield Method is explained. For example:
(i) The purpose of the promise chain and its ability to give easier, more comfortable and more reliable personal control.
(ii) The promises will be added to the program in a “chain-like” manner:
(iii) Each promise will be added and numbered in the order that it was made.
(iv) Alternatively, the promises may be added by type of behavior in a chainlike manner.
(v) If the participant breaks a promise, he or she is not just breaking the one promise but his or her entire promise chain, which will have a negative effect on his or her Promise Score (as shown in FIG. 13) and may be irreversible.
(vi) This is a powerful incentive to motivate him or her or her to keep his or her score as high as possible.

Referring to FIG. 6, the Shielder participant will be walked through to make a first promise of his or her promise chain (whenever “promise” is used, below, it means “promise added to his or her chain of promises):
(i) The Shielder participant may be asked to start his or her chain at a time when he or she won't be eating.
(ii) The Shielder participant is directed (through explanation or illustration) to type or select a promise in the “Promise Entering” screen in the promises-tracking device of the invention.
(iii) For a first promise, the Shielder participant is usually prompted to type in or enter a specific promise, such as “I promise not to eat anything—for 15 minutes.” A cursor may appear after “I promise,” to help him or her start each promise.
(iv) Alternatively, the Shielder participant may only have to select or fill in spaces to complete a promise, such as “I promise to only have 14 of a roll for 15 minutes.”

Each promise may require that an item, quantity and/or term be specified. For example, “I promise to only eat Vi (quantity); of a roll (item); for 15 minutes (term).

The Shielder participant may not be able to enter a promise unless it the required specifications. It is explained to the Shielder participant that he or she must push or tap an “enter” key (or “Add Promise to Chain” key) to add a promise to his or her promise chain.

It is explained that once he or she adds a promise to his or her chain, he or she is responsible to keep it, “no matter what.”* (except when excused by the rules,” which are described below). The Shielder participant is directed to push the “enter” key. Once he or she pushes the enter key, the promise is designated as a viable promise on the screen 13 with a number (1), for example, next to it. There may be an animated illustration showing that a link has been added to his or her promise chain and he or she is informed that this has started his or her promise chain and is congratulated.

It should be noted that the promises-tracking device 1 may be able to remember his or her promises so that once he or she begins typing them, the promises-tracking device 1 will finish or partially finish typing it for him or her or her; and/or he or she may be able to select past promises; and/or there may be a menu of suggested promises for him or her or her to select from.

Referring to FIG. 6, the Shielder participant is then taught the basic rules of making promises as described above, such as:
(a) A rule that the promises he or she adds must be easy for him or her or her to keep.
(i) The Shielder participant is instructed in how to make promises that are “easy” for him or her or her to keep. This is no different than developing many other kinds of skills, such as ice skating. A new skater doesn't recklessly push himself out onto the ice. He or she starts out cautiously, getting a feel for the ice, and stays within his level of ability. Similarly, the Shielder participant is taught to make promises that will be extremely easy for him or her or her to keep, at first; and is given suggestions for making easy promises, such as promising to only have half of a roll for 30 minutes. As the ice skater uses feedback from his experience, and has a healthy “fear” of overdoing it, so does the Shielder participant, who gets a feel for making promises, and knows that he or she could “ruin his Promise Score,” or even fail if he overdoes it. This helps him or her or her stay within his or her level of comfort.
(ii) Examples of very easy, yet fun and effective, promises to use until he or she gains greater skill and confidence are provided, such as promises to “lock in” his or her intentions; short “time out” promises (the same kind of “time outs” that are effective for controlling children); and the “3 more bites” technique (when he or she can't stop eating from a bag of chips, for example, he or she can make a promise to stop after 3 more bites).
(iii) Techniques for making easy promises are provided. For example, he or she is taught that reducing the term of a promise will make it easier to keep (promising not to have a second roll for 15 minutes is much easier than promising not to have another roll for 6 hours;
(b) A rule that the promise he or she adds must be short-term (usually less than 6 hours).
(i) It is explained that promises have a length of time assigned to them, but they don't have to have an exact time. For example, if he or she is standing at a checkout counter at a grocery store, he or she can promise not to eat anything from his or her grocery bags until he gets home and puts the food away.
(ii) Some promises may be longer, such as a promise not to have “any” or “any more” of a specific food.
(c) A rule that states that once he or she adds a promise to his or her promise chain, he or she must keep it “no matter what.” (except when excused by other rules, as described herein.)
(d) A rule that he or she is completely free of the obligation of each promise after keeping it.
(e) A rule that excuses him or her or her from keeping any promise which is unsafe or unhealthy to keep.
(f) A rule that he or she may make more than one promise for the same time period; and promises can overlap. For example, he or she can make a new promise while he or she is keeping another or others.
(g) A rule that he or she can upgrade or improve his or her promises. For example, if he or she has promised to only have two cookies, he or she can change the promise to just one cookie or no cookies.
(h) A rule that he or she can increase the term of promises.
(i) A rule to only make promises to do better. For example: can't make promises to eat more than usual. There may be exceptions to this, usually with doctor's supervision.
(j) A rule that if he or she knowingly breaks a promise, the penalties are:
(i) The participant has broken his or her chain and his or her Promise Score is permanently marred.
(ii) The participant may lose all privileges of adding promises to his or her promise chain for 24 hours (or other term).
(iii) The participant is warned that he or she is putting his or her ability to program his or her mind to help him or her or her keep his or her promises in jeopardy, since he or she is teaching his or her mind not to take his or her promises seriously, which could cause him or her or her to permanently lose the ability to reliably program his or her mind.
(k) A rule that excuses him or her or her from being penalized for not keeping promises that he forgets to keep, on the grounds that he or she is “innocent.”
(l) A rule that states that if he or she is in the act of “innocently breaking” a promise, he or she can continue with the “illegal” behavior until it is comfortable for him or her or her to stop. For example, if he or she is eating from a bag of potato chips and suddenly remembers he or she promised to only have ten chips, but has had more, he or she will be allowed to make a follow-up promise to have 3 more chips, so that it is comfortable for him or her or her to stop. This “pressure releaser” rule is necessary because the primitive mind is so powerful that it could force him or her to break such promises, causing him or her to “innocently” fail.
(m) A rule that he or she must get busy doing other things during his or her promises, such as he or she can't just sit and watch the clock.

Referring to FIG. 6, the Shielder participant is instructed and motivated to add more promises to his or her chain:

The Shielder participant is encouraged to add more promises to his or her chain.

The Shielder participant is motivated to add more promises to his or her chain. To motivate the Shielder participant, he or she is told the benefits of making promises and how it can help him or her obtain his or her goals:
(i) By keeping his or her promises easy and short-term, it's possible and even easy to keep them.
(ii) After keeping each promise, he or she is completely free and relieved of all restriction, which relieves stress and feelings of deprivation. This is a great improvement over most diets, which require him or her to be deprived for days, weeks, months or even years on end!
(iii) Each promise “locks-in” better behavior.
(iv) Any promise to do better is an improvement.
(v) If he or she makes a lot of little improvement promises, it can add up to real progress towards his or her goal. The promises-tracking method gives many ideas for making very easy, yet very effective promises.
(vi) Any time the Shielder participant feels strong, he or she can “lock-in” a promise for better behavior.
(vii) The Shielder participant can make promises for any eating improvements that he or she can think of—the possibilities are limitless.
(viii) The chain of promises has a doable, lasting quality that can finally make it possible for him or her to succeed at weight loss. (All Shielder participants of the test group said that they could easily maintain their control indefinitely—even those who gave up previously on diet after diet.)

Referring to FIG. 6, the Shielder participant is taught what to do if he or she breaks a promise. Actual penalties are listed in the rules.

According to the preferred embodiment of the present invention, the excused broken promise button 113 and the unexcused broken promise button 114, as shown in FIG. 1 to FIG. 3, are provided for the Shielder participant to input by touching or pressing if he or she breaks a promise.

The excused broken promise button 113 is configured for the Shielder participant to input when he or she breaks a promise that was excused per the rules. There is no penalty for breaking a promise if it is excusable by the rules, such as if he or she “forgets” to keep a promise, although the software will keep track these and prompt him or her to take action to reduce these occurrences, if they happen too frequently. Such action might be to write down his or her promises to help him or her remember them.

The unexcused broken promise button 114 is configured for the Shielder participant to input when he or she breaks a promise that is not excused or innocent per the rules. There are penalties for breaking promises being unexcusable by the rules, such as losing privileges of making promises for 24 hours, negatively effects on his or her promise score, and etc.

Please note that, according to the promises-tracking method of the invention, it is assumed that all promises are kept unless the Shielder participant inputs by touching or pressing one of the excused and unexcused broken promises buttons 113, 114. In the clinical trials, almost all Shielder participants could easily remember when they broke a promise.

It is worth mentioning that the promises-tracking device can be a program operated electronic device 1 custom made for processing the promises-tracking method, wherein the time setter 111, the activator 112, the excused broken promise button 113, and the unexcused broken promise button 114 are configured as physical buttons of the input device. An electronic device, such as smart phone, tablet and handheld computer with different operation systems, can be programmed to function as the promises-tracking device 1 by downloading an App. For example, the display of “promise time” will be shown on the touch screen of the smart phone. The time setter 111, the excused broken promise button 113 and the unexcused broken promise button 114 can be programmed as touch buttons respectively provided on the touch screen of the smart phone. The home key of the smart phone can be programmed to be the activator 112.

The time setter 111 is provided for the Shielder participant to selectively adjust the promise time. As mentioned above, the excused broken promise button 113 is configured to be used if the Shielder participant breaks a promise, but was excused by the rules. The unexcused broken promise button 114 is configured to be used if the Shielder participant breaks a promise and is not excused or innocent per the rules.

Furthermore, the activator 112 can be an independent element such as the ring as shown in FIG. 7 configured to be wirelessly linked to the program operated electronic device 1 via any wireless link such as Bluetooth, Wi-Fi, infrared, or Radio frequency, such that when the activator 112 is actuated, the program operated electronic device 1 will be notified to start the promise and seal it to the promise chain.

It is worth mentioning that the present invention may not notify the Shielder participant once promise times are up, to help the Shielder participant extend his or her abstention (the non-eating time) as long as possible. For example, if fat Albert has made a promise to only have one small piece of pie for 3 hours, reminding him or her when the three hours is up is like ringing the dinner bell! If he or she is not reminded, he or she may not think about having more pie for another hour or longer, which can be beneficial. However, the promises-tracking device 1 may notify the Shielder participant when certain kinds of promises are over, if desired, such as promises to do exercise.

When the Shielder participant checks the promise time, the screen 13 of the promises-tracking device 1 can be configured to display the initial promise time that the Shielder participant made and the required term of each promise. Alternatively, the abstention time of the Shielder participant, i.e. the amount of time the Shielder participant actually abstained, can also be displayed, such that an “overtime monitor” is provided to display his or her total time extensions, to encourage him or her or her to extend the behaviors of his or her promises.

Referring to FIG. 6, the Shielder participant is assisted in making a strong commitment to keeping the promises he or she adds to his or her chain. Any or all of the following methods may be used, where “Promises” refers to the promises he or she adds to his or her chain of promises:
(a) The Promise Score Display, which displays his or her Promise Score (ratio of promises kept to those broken), as shown in FIG. 13.
(i) The program informs him or her or her that every promise he or she adds to the promise chain will be accounted, which will record whether or not he or she keeps each promise and give him or her a ratio score of promises made to promises kept.
(ii) He or she is encouraged to keep his or her score as high as possible. As one way to encourage him or her or her, he or she may be told that he or she cannot reset his or her score or start over, so he or she can permanently damage his or her score by breaking any promise. Also, his or her score may be for the lifetime of his or her use of the promises-tracking device and the method thereof; or for a specific term; or for a certain number of promises; or it may be reset with a code given to his or her doctor, instructor, or coach; or to him or her under “excusable” conditions, such as a major misunderstanding that caused him or her or her to break promises which is now understood.
(iii) The “Promise Score Display” is described to him or her or her, including:
(1) The total number of promises he or she has added to his or her chain.
(2) The number of unexcused broken promises.
(3) Optionally: the number of the excused broken promises.
(4) The program will recalculate and display changes to his or her promise score.
(iv) His or her Promise Score(s) may be posted on the face of his or her app, other devices and on social network programs, to further motivate him or her to keep his or her score as high as possible.
(b) By describing the benefits of keeping his or her promises: If he or she reliably keeps his or her promises, he or she will be able to reliably program his or her mind to help him or her or her keep his or her promises, for all the benefits of the promises-tracking method.
(c) By reminding him or her that it's easy to keep his or her promises:
(i) He or she is reminded that he or she doesn't have to make any promises; he or she only has to keep the ones he or she decides to add to his or her chain; and he or she only has to make easy promises.
(ii) He or she may be asked to admit to himself or herself that he or she can keep all of the promises he or she adds to his promise chain, unless excused by the rules.
(d) By teaching the Shielder participant that there is nothing stressful or unnatural about committing to do things. People easily and naturally make commitments throughout life. They simply make a decision, which can be easily and comfortably maintained as long as it is easy enough to do and the consequences (having too much to lose to break the decision) are sufficient to maintain it. Examples include the commitment to always add detergent to loads of washing, to avoid stains and dirty laundry, and the commitment to drive in the legally designated lane, to avoid getting in auto accidents.
(e) By asking the Shielder participant to “decide” (or make up his or her mind) to keep his or her promises.
(i) He or she is taught that “decisions” can program the mind—although it takes having “too much to lose ” to maintain the decision as described above.
(ii) The benefits of deciding (making his or her mind up) to keep his or her promises are explained to him:

As long as his or her mind is made up to keep his or her promises, so that he or she fully intends to keep them, this will enable him or her to cross the “threshold of sureness” that is required to program his or her mind to help him or her keep them. For example, Yes, it's a circle: one must create the “sureness” that he or she will follow the behavior that he or she wants to program his or her mind to help him or her follow.

It is explained to the Shielder participant that programming his or her mind to help him or her keep his or her promises will make it much easier for him or her to keep his or her promises and give many additional benefits as described above.
(f) By teaching the Shielder participant that the consequences of breaking promises must be strong enough to keep him or her comfortably and confidently sure that he or she will intend to keep every promise he or she adds to the device (just like the consequences of losing his or her credit keep him or her comfortably and confidently sure that he or she must make every car or house payment). After experiencing the incredible boost that super willpower gives, losing it often becomes too much for the Shielder participant to lose! Then he or she is taught the vital concept that he or she will lose it if he or she even intends to allow exceptions in keeping his or her promises, even occasionally (for it's not possible to intend to allow exceptions and be sure at the same time). This keeps him or her comfortably and confidently sure that he or she must keep every promise he or she adds to the promises-tracking device 1.
(g) by teaching the individual the consequences of breaking promises:
(i) Each time he or she breaks a promise, he or she is teaching his or her mind not to take his or her promises seriously. Once his or her mind learns this, he or she can lose his or her precious personal control permanently. For most people in the program, this is “too much for them to lose” and a very strong deterrent for breaking promises. Please note that the 1% in the clinic who allowed themselves to break promises had to start fighting their minds to keep their promises within a couple of weeks—and it soon became extremely difficult and unpleasant for them to keep them! But the 99% who simply made up their minds to keep their promises maintained super willpower for keeping them indefinitely—even those who were tracked for years.
(ii) If he or she even seriously intends to break promises, he or she has already lost the sureness required to program his or her mind, since it's not possible to intend to break promises and be sure that he or she won't at the same time. Therefore, if the individual values the ability to program his or her mind to help him or her or her follow behaviors that his or her primitive mind would otherwise fight, he or she has to make up his or her mind to keep his or her promises, so that he or she intends to keep them.
(h) By giving the Shielder participant assignments to help him or her or her experience the ease, comfort and control of following his or her plans while his or her mind is programmed to help him or her follow them.
(i) By informing the Shielder participant that it's his or her responsibility to keep his or her promises, so that he or she feels responsible to take care when making and keeping them.
(j) By pointing out that keeping his or her promises is a small price to pay for the benefits of being able to program his or her mind to help him or her keep his or her promises.
(k) By teaching him or her that the Shield Method uses the same method that the mind naturally uses for easy, comfortable, reliable personal control; and that man-made methods are not as effective, since they force people to fight their minds. The current obesity epidemic is proof that failure is likely with current methods, like diets, that require people to fight their minds in order to try to stick to them.
(l) By teaching the Shielder participant that he or she probably won't be able to succeed at his or her goal if he or she has to fight his or her mind because of how powerful the mind is. Failure is almost certain (or much more likely), when a person has to fight his or her mind to follow a plan, because of it's powerful mind-tools, whereas success is nearly guaranteed (or much more likely) when his or her mind is programmed to help him or her follow his or her plan.
(m) By reassuring the Shielder participant that he or she isn't expected to be “perfect,” to set him or her at ease.
(i) He or she is told that it will not always be “extremely easy” to keep every promise he or she adds to his or her chain. Sometimes it will be a little difficult not to have that second delicious roll that he or she promised not to have for 30 minutes after he or she finishes the first. This is why it's important for his or her promises to be easy and short-term, because he or she may have to use a little ordinary willpower to keep This promise. But since the promise is easy and short term, he or she only has to “tough it out” for a short time—not days, weeks or years, as he or she would on a diet or other weight loss method. Keeping the easy promise helps him or her stay in control; and afterwards, he or she is completely free to have more.
(ii) He or she is encouraged to have just a little more for another 15 minutes. This and other techniques described in the “Technique and Tips” section, below, help him or her stay in control even after his or her promises are over.
(iii) He or she is told that if he's like most “Shielder participants,” he or she will occasionally break some promises—nobody is perfect. In which case, he or she must pay the 24 hour penalty, per the rules. It is found that depriving Shielder participants of their precious personal control for 24 hours is the most effective penalty for helping them learn to appreciate it.
(n) By pointing out that the commitment doesn't have to be constant. We sometimes daydream and flirt with ideas of breaking all kinds of commitments, such as missing a day of work. But if we conclude that we have too much to lose to skip the day, the desire to keep our job will kick in and program our mind to help us go to work. In the same way, the Shielder participants sometimes think about breaking a promise—it's allowed, and completely natural! But once we conclude that we can't, because losing our precious control is too much for us to lose, our mind will kick in and help us keep the promise. The Shielder participant is encouraged to make a long-term decision to keep his or her promises, like he or she may make a long-term decision to take the trash out every week, to avoid having to fight many mind battles about it.
(o) One of the most effective strategies in the clinic was asking individuals to make “keeping their promises” a part of their character—a part of “who they are,” so that once they make a promise it is tied to their character. This is one reason why we refer to the “behavior plans” as “promises.” The individual often develops a deep personal commitment to keeping his or her promises, to the point that it becomes part of his or her moral fiber and is part of what defines him. The individual then has a personal stake in keeping them and feels that he or she has too much to lose to break them, since his or her commitment adds so much value (personal control) to his or her life.
(p) By creating Categories and/or Levels that he or she may attain.
(i) As his or her knowledge and skill progresses, we say that his or her “Shield” goes from paper (the least amount of protection), to cloth, to wood, to tin, to metal and finally to sterling silver. (The Sterling Shield is the highest level, giving the greatest comfort and protection.)
(ii) Three categories are used, based on how much more the person eats after his or her promises.
(iii) These levels and categories may be displayed on the screen 13 of his or her promises-tracking device (iPhone, etc.) or on social networks such as “Facebook”, as shown in FIG. 15.
(q) By describing how his or her “chain of promises” makes it more difficult for his or her mind to fight his or her personal plans:
(i) Without the promise chain, his or her mind is free to FIGHT the behavior plans he or she makes by:

Using the “procrastination rationalization”: he or she can easily rationalize that he or she can “make up” for breaking any plan later. For example, if he or she makes a plan to exercise for an hour “today,” his or her mind will be free to FIGHT his or her plan by helping him or her rationalize that he or she can skip his or her exercise “today,” and make up for it “later.”

His or her mind will also be free to fight his or her plan by helping him or her rationalize that he or she has good enough excuses to break his or her plan or that he or she has “more important” things to do.
(ii) With the chain of promises, his or her mind cannot so easily fight his or her plans because:

By linking every promise to a chain, it's not just one promise he or she is breaking or one misdeed that he or she can easily make up for, but his or her entire chain—which he or she gives him or her the ability to program his or her mind to help him or her follow plans that his or her primitive mind would otherwise fight. In other words, breaking a promise won't just result in missing one day of exercise that he or she can rationalize he or she can make up for. It puts his or her chain, his or her ability to program his or her mind, his or her control and his or her ability to succeed in jeopardy. For most Shielder participants, that's just too much to lose.
(r) By using the “Locking Mechanism”, as shown in FIG. 5. Most of the above methods for creating a commitment to keeping promises contribute to the “Locking Mechanism,” as described above.

The following are “Locking Mechanism” concepts:
(i) Since it is EASY, therefore POSSIBLE, for the individual to keep his or her promises;
(ii) Since he or she must INTEND to keep every promise he or she adds to his or her chain in order to program his or her mind (referred to as activating “Mental Shields” in the original application) consistently;
(iii) Since he or she values (and wants to PROTECT) his or her ability to program his or her mind consistently, for the benefits of increased success rates, increased comfort, ease, self-esteem, progress and decreased stress:
(iv) The result is: he or she makes his or her mind up not to make any promises unless he or she INTENDS to keep them, which enables him or her to program his or her mind reliably and at will. (In other words, he or she gets “locked-in” to keeping his or her promises.)

It should be noted that there has been some confusion about the “absolute sureness” spoken of in the original application. This happens at the moment a person “decides” to keep (or go ahead with the behavior of) his or her promise—it does not mean that the individual is “always” sure that he or she will keep every promise, any more than he or she is always sure that he or she will go to work every day. This sureness is the “go-ahead” by the ego division of the mind, once a decision is made to keep a promise (as was described in the scientific explanation of the original application). The sureness may waver and only become firm seconds before actually keeping a promise.

Referring to FIG. 6, committing to keep his or her promises through the promises-tracking device and the promises-tracking method of the invention gives the Shielder participant a reliable personal control. Now he or she is taught the “Promise Techniques” as described above, which enable him or her to use the “leverage” of his or her reliable control for easy, comfortable and consistent control.

The Shielder participant is taught that short-term promises can be very effective. For example, 15 minute promises can be very effective, for the following reasons:
(i) Stopping for 15 minutes stops the hand-to-mouth reflex and fools his or her mind and body into thinking he or she is done eating. This makes it easier to eat less.
(ii) Taking a 15 minute break helps him or her stay in control and gives hot desires a chance to cool down. There are 900 seconds in 15 minutes. If he or she feels strong for even one of those seconds, or realize that what he or she was eating wasn't very good, he or she can lock in a promise to stop. Before, he or she would have eaten the whole thing and then regretted it.
(iii) Waiting 15 minutes allows time for food to reach our stomach and register sensations of fullness and satisfaction.
(iv) After the 15 minutes, he or she usually feels refreshed instead of stuffed or sick and ready for a nap—and he or she starts to prefer feeling This way after eating even when he or she was certain that he or she would.
(v) After 15 minutes, his or her food may be less appealing (cold, wilted, etc.) and This makes it easier not to have more. (After a while, this doesn't matter because most Shielder participants “grow out ” of having more—or they are usually fine with just a little more.

The primitive mind puts up less resistance to shorter promises because instant gratification is obtainable sooner. So, these promises are very easy to make, yet so effective at keeping them in control that we say they have “already won” by making them.

The Shielder participant is taught techniques that enable him or her to use the methods that the primitive mind uses “in reverse,” for easy, comfortable, reliable and effective personal control.

The primitive mind craves instant gratification, so it endeavors to make “feast now, fast later” deals with the over-eater, causing him or her to eat excessively when he or she eats. Such a deal is, “If you eat another big piece of pie now, you can make up for it by dieting all next week.” This is how his or her primitive mind has been fooling him or her into eating too much. These deals are unhealthy (it's unhealthy to eat so much) and unreliable (since the primitive mind is likely to fight them and work for another quantity deal the very next time he or she is tempted). But now that the Shielder participant has the ability to add promises to his or her chain that he or she won't ordinarily break, he or she can reverse This deal by making “fast now, feast later,” promises: “If I eat just half a piece of pie now, I can have more, if I want, in 30 minutes.” In This way, he or she reverses the trick played by his or her primitive mind so he or she eats less. Using such promises to delay quantity is much healthier and helps him or her stay in control, for all the reasons described above. Also, the rules of making promises stipulate that he or she must get busy doing other things during his or her promises (he or she can't just sit and watch the clock). And he or she is encouraged to make a promise to have just a couple more bites if he or she really wants more after the 30 minutes. The leverage, rules and reliable control that his or her chain of promises gives helps him or her stay in control and even to lose his or her fear of losing control, reducing stress and anxiety greatly.

The Shielder participant is taught techniques for “consistent” control:
(i) With one important technique for consistent control, he or she promises to eat half as much food each time one eats for 15 minutes (the Hl5 technique). In other words, he or she will make promises to eat no more than half as much (put half as much on his or her plate) and wait 15 minutes before eating more (the 15 minutes starts after eating the half).

The Shielder participant may be assisted in committing to use the Hl5 technique every time he or she eats; using many of the same principles used to commit to keeping his or her promises. This helps ensure that he or she makes “our best promise” every time he or she eats, which can give him or her more consistent control.

A benefit of committing to the Hl5 technique is that it simplifies the entire program for him, since this is the only promise he or she needs to make. Committing to this technique ensures that he or she will make a great promise every time he or she eats and usually results in eating less than he or she would have. Of course, as he or she is warned: for the over-eater, trying to eat half may feel like “unbearable deprivation” unless his or her mind is programmed to help him or her do so.

The following are special rules and tips for this technique, such as how to estimate and eat half as much in every kind of situation:
(i) The 15 minutes starts after he or she has finished eating.
(ii) Put the rest of the food away, out of sight, before starting to eat. This helps the Shielder participants get involved with non-eating activities. People often forget to have more for hours.
(iii) If the Shielder participants want more, ask themselves how much they really want. Often a couple more bites is all we need. If so, be sure to lock it in with a promise.
(iv) Eat in a civilized manner. This helps the Shielder participants enjoy their food more than wolfing down excessive amounts without really tasting it; which helps them learn to enjoy less food more than excess.

It is worth mentioning that the Shielder participants are preferred to be taught a unique terminology associated with the Shield Method, based on the word “Shield,” which can be used in the traditional sense, to mean “protect,” or to mean “program” or “super personal control.”

The Shielder participant is taught, for example, “You will be able to Shield (or program) your mind to make you comfortable with just one scoop of ice cream—and This will Shield (or protect) you from feeling deprived without more. Your plan to have just one scoop will then be Shielded (or protected). You will be able to use your Shield (your super personal control) to easily and comfortably lose weight and to achieve many other personal goals. A Mental Shield is a programmed state of mind, which is acting to protect a person, plan, behavior or a belief. In addition, those who learn and use the Shield Method are called Shielders.”

The promises-tracking method of the present invention is also arranged to teach the Shielder participants to distinguish between the 2 types of willpower:

Ordinary willpower: A person has to fight his or her mind in order to do something. An example is sticking to a traditional diet. The dieter has to fight his or her mind as it tries to tempt him or her to eat the foods or quantities that are not allowed on his or her diet. This kind of willpower is difficult and stressful to use and it is extremely unreliable. Most dieters cheat on their diets and fail daily.

Super willpower: A person doesn't have to fight his or her mind to do something. In fact, his or her mind helps him or her. This makes it super easy and comfortable to do, and gives super high success rates. An example is driving on the correct side of the road. People don't have to fight their minds to do this; their minds help them, giving a super success rate—most people always stay on their side of the road.

There are three divisions of the mind, namely:
(i) The primitive id (inner child) division of the mind.
(1) Information about the nature of the id, which is ruled by the “pain and pleasure” principle.
(2) Information about the “mind tools” that the primitive mind can and does use to stimulate people to act to obtain things that they find desirable.
(3) How the primitive mind and/or ego uses the “mind tools” when the mind is programmed to help him or her resist temptations.
(4) Techniques which use the actions of the primitive mind for enhanced behavior control.
(5) A cartoon character named “Id the Dragon” may be used to represent the primitive mind and to illustrate lessons and concepts and for promotional purposes.
(ii) Information about the ego (inner adult) division of the mind.
(1) Information about the nature of the inner adult, which can over-ride the inner child when the individual feels that he or she has “too much to lose.”
(2) A cartoon princess character may be used to represent the ego division of the mind and to illustrate lessons in various ways.
(3) How the inner adult can gain better control of the inner child, using the leverage of the chain of promises.
(4) A cartoon character named “Judge Ego” may also be used to represent the ego division of the mind and to illustrate lessons and concepts and for promotional purposes.
(iii) Information about the super ego division of the mind.
(1) Information about the nature of the super ego, which serves as a person's conscience and reservoir of information about why a person should and should not behave certain ways.
(2) A jury of cartoon characters may be used to represent the super ego division of the mind and to illustrate lessons and concepts and for promotional purposes.
(3) Information about nutrition, exercise and/or metabolism.

The promises-tracking method is arranged to track and make the Shielder participant aware of patterns, problem times and progress and to periodically give other motivational and encouragement messages, such as notifying him or her that he or she can make a better promise in a certain situation. The promises-tracking method is also arranged to give reminders and suggestions, such as reminders he or she has scheduled and suggestions to make specific promises in certain situations, and to notify the Shielder participant if he or she is breaking a rule; if he or she is in a “danger” time period or situation in which he or she typically has problems over-eating; if he or she has a misunderstanding of a basic concept of the program, and etc. Further, according to the promises-tracking method, lists or checklists of promises are provided for the Shielder participant to choose.

The promises-tracking method is arranged to teach specific techniques and tips for creating new habits, including:
(i) specific techniques and tip for accomplishing “do ” behavior goals, such as chores and special projects. One “do” behavior technique is called the DI5. The Shielder participant makes a promise to do something just for 15 minutes. Not only will he or she get at least 15 minutes done.
(ii) specific techniques and tips for accomplishing other “don't” behavior goals, such as don't bite his or her nails.
(iii) Effective techniques such as the delay techniques, for example stopping for 15 minutes. 15 minutes stops the “hand to mouth” reflex and gives food time to register feelings of fullness and satisfaction, and etc.

In view of above, the promises-tracking device and the method thereof substantially enable Shielder participants to make promises, under the supervision of a psychologist, to endure situations that they find difficult; so that they can experience it while their mind is programmed to help them endure it, rather than fighting their efforts to do so.

The promises-tracking method of the invention is unique in that it enables the Shielder participants to program their minds for any behavior they add to their chains, wherein the possibilities are limitless.

It is worth mentioning that many conventional methods may be improved by the Shield Method of the present invention. For example, the “time out” can be improved with the chain of promises of the invention. It is even found that naps can be more restful by making term promises, since people don't have to fight mind battles that urge us to keep getting up. One experienced Shielder participant said that he or she was able to use “delay” promises to cure himself of masturbating whenever he or she was alone.

Other specific techniques, as well as tips, are taught through the promises-tracking method of the invention for controlling the primitive mind, including:
(i) “inspiration lists” or “checklists” of things to do, ideas and goals, designed to inspire the Shielder participant to make promises that will help him or her accomplish his or her goals.
(ii) lists of new “dream habits” that the Shielder participant may choose to establish.
(iii) a promise calendar, which shows the promises the Shielder participant is scheduled to keep. The Shielder participant may be able to schedule promises directly into the calendar.

The promises-tracking method may also provide a capability for the Shielder participant to log his or her daily activities and/or activities related to his or her goals, such as what he or she eats, and a capability for the Shielder participant to log his or her “Guilt's” and/or “Gloats”: things he or she feels “guilty” about doing and/or things he or she feels “proud” of doing.

The promises-tracking method may provide a Trouble Shooter that may be used if the Shielder participant has a problem or question. The final result is that the Shielder participant is able to maintain the sureness that he or she will keep the promises he or she adds to the promises-tracking device, with a high degree of reliability, so that he or she can program his or her mind “at will” to help him or her follow the behaviors of his or her promises, easily, comfortably and with a high degree of reliability. Highly documented and audited clinical tests showed that all Shielder participants were able to create and maintain the sureness to keep the promises they added to their promise chains. By processing the promises-tracking method, the Shielder participants are enable to program their minds so that it significantly reduced having to fight their minds to follow improved behaviors.

In view of above, the promises-tracking device and method thereof of the present invention enable the Shielder participants to reliably and consistently activate and effectively use the natural “Mental Shielding” ability of the mind so as to reliably and consistently program their minds to help them easily and comfortably resist temptations that would otherwise be very difficult and unpleasant for them to resist. It can significantly increase success and self-esteem, while decreasing stress, illnesses and deaths. It can even quickly and permanently improve preferences and habits. The promises-tracking method of the invention is an important advancement which can dramatically reduce problems resulting from a lack of personal control.

None of the prior art proposes the idea of creating mental sureness as a method for programming the mind for enhanced behavioral control; or makes creating mental sureness the specific goal of a method; or suggests finding a way to use. This natural ability that the mind uses to help people resist temptations; or talks about the incredible advantages that the present invention can give. There has never been an effective or practical means of enabling people to activate this ability of the mind for personal control applications “at will” before. Indeed, it was presumed to be impossible, which is why no-one ever tried. These concepts disclosed in the present invention were never even discussed-except perhaps by the military, to prepare soldiers mentally for combat situations.

The present invention created a mental sureness to keep our promises. It is noticed that our minds were not fighting the behaviors of our promises. In fact, it was helping us keep them. This was a very useful, yet unexpected and unobvious result. Each of the medical doctors and psychologists who participated in the clinical trials also certified in writing (and also on video tape) that adding a promise to their chain did, indeed, “set” (or program) their minds to keep it, making behaviors that would have been difficult-such as eating much less and stopping after a promised limit-much more easy and comfortable to accomplish.

Even if someone had thought of the idea of programming the mind by creating mental sureness, how would they go about creating it? In the military, months of intensive, secluded training are necessary and the programming is directed specifically towards readiness for combat. The promises-tracking method of the present invention is the first that create this sure mental state instantly, easily, reliably, comfortably, “at will,” any time or place for personal control applications.

According to documented clinical trials of the present invention, once reliable personal control was established, over-eaters automatically started to go through some very positive transformations. Feelings of depression and helplessness were naturally replaced by feelings of accomplishment and power. In addition, the participants actually began to prefer less food to their old excesses. This is a reversal of negative transformations that they descended through as they lost control over what and how much they ate.

It is appreciated that possibilities for child rearing applications are numerous. Overweight children can process the promises-tracking method (Shield Method) of the invention to lose weight and/or gain more personal control. Parents can use the system to better understand their children and learn more effective ways to control them, and establish better behavior Shields in them.

Numerous embodiments are foreseen in the field of psychology. With modifications, The Shield Method of the present invention could help treat addictions and many psychological disorders, such as obsessive and/or compulsive disorders. The Shield Method of the present invention can give people a way to control their behaviors and decrease the frequency and intensity of their repetitive or other undesirable behaviors. The present invention can enable people to experience new behaviors in a more non-threatening and comfortable way so that they can more easily learn to prefer better behaviors and change their habits. The Shield Method of the present invention significantly decreases stress while increasing control and feelings of self-esteem, which are extremely important in the field of psychology.

The present invention may benefit people in the teaching and people management fields. The present invention can provide people more effective ways of understanding and controlling behaviors, stress, increasing ease of tasks, and etc.

It is worth further mentioning that the present invention could be used to increase group performance, such as to reach goals set by a company or group, such as for relationship improvement like family, spouse, and other.

The present invention can have very important uses in the field of criminal science. Offenders have never had a way to control their behaviors other than with willpower, which gives very low success rates. Shield Method programs could help people better understand the criminal mind, provide more effective ways of changing behaviors, and etc. Since the present invention can change habits permanently, many previous systems that weren't very effective may now be effective. For example, weight loss drugs or clinics which can take weight off more rapidly may now work, because people have changed their eating habits and won't regain the weight afterwards. This is supported by medical science. According to Guyton's Textbook of Medical Physiology, the body's fat stores are maintained like a bank account. After a person gains the weight, it is like they have a large balance. Then, they only have to pay the monthly maintenance fee in order to maintain the balance. In other words, an overweight person does not have to overeat to maintain their weight. Doctors now frown on quick weight-loss programs before, because habits weren't changed and patients were likely to gain the weight back as soon as they went back to their old eating habits. Shielders who change their habits may be expected to keep the weight off for good on one of these programs.

It is appreciated that a camp or retreat which would teach the Shield Method and offer rapid weight loss and/or other beneficial services simultaneously, such as to teach health concepts and increase metabolic rates, would be ideal.

Also, computer programs, which can teach, test and/or motivate people in the use of the Method. Computerized (or other) games or practice devices that help participants increase their skill at forming and using Mental Shields. Computerized (or other) systems which can be used as a “physical activation signal,” remind participants of the promises they have made, when to make a promise, the rules, techniques, motivational or educational information.

Computerized device and the like can be programmed to form the promises-tracking device for the Shielder participants to process the promises-tracking method to produce the “sureness” state of mind required to activate Mental Shields.

The present invention may be used in reverse for “reverse psychology” applications, such as to have a person experience a negative behavior with which to compare a positive one. This application is foreseen to be useful for people suffering from depression or to help people understand the negative aspects of behaviors whenever there may be a beneficial use.

It should be noted that the preferred “naturally thin eater” model is optional. Any other model of eating can be used in the embodiment for weight loss. Optionally, predetermined steps of the preferred embodiment of learning the Shield Method may be eliminated. For example, people can discover the benefits, uses and their lack of Shields without being specifically taught. Also, new permanent Mental Shields do not have to be established for positive results. The Shield Method allows “new use” of all of the “prior art” techniques and uses listed in this application, wherein when it is incorporated with the promises-tracking method of the invention, they are suddenly much more functional, effective and give a dramatically different and improved result.

Although the description above contains much specificity, these should not be construed as limiting the scope of the invention but as merely providing illustrations of some of the presently preferred embodiments of the present invention. Many additional applications can help people utilize the benefits of the Shield Method. Although the preferred embodiment as described above uses weight control application as an example, it is appreciated that embodiments for substance abuse control, such as for smoking, alcohol or drug use could be similar to the weight control application of the present invention. Steps could easily be modified so that the amount and frequency use of these substances would be reduced and eventually eliminated, if desired. Embodiments for “do” behaviors, such as to exercise consistently or complete a project, would also be similar to the weight control application of the preferred embodiment of the invention.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A promises-tracking device which is a program operated electronic device for forming a promise chain of a plurality of viable promises of a participant and a promise score for tracking the plurality of viable promises of the participant, comprising:
at least one processor;
an input device comprising an excused broken promise button, an unexcused broken promise button, a time setter configured for setting promise times of the plurality of viable promises, an activator configured for the participant to generate a physical activation signal when each of the plurality of viable promises is made by the participant, wherein the excused broken promise button and the unexcused broken promise button are configured to be selectively inputted for an input of a broken promise when one of the plurality of viable promises is broken;
at least one memory module stored program to instruct the at least one processor to receive a plurality of viable promises inputted by the participant through the input device in a chainlike manner and store the plurality of viable promises to form the promise chain on the at least one memory module; and
a screen configured to display the physical activation signal generated through the activator of the input device to notify while the viable promises is sealed in the promise chain, the setting promise times, in response to the plurality of viable promises of the promise chain in a numbering order in the memory module, being set via the time setter, a notification when the promise times are up, and a level of sureness of the participant graphically for the participant to view and periodically for monitoring the level of the sureness of the participant, wherein when the unexcused broken promise button is selectively inputted for the input of the broken promise while one of the plurality of viable promises is broken, the promise chain is then broken to have a negative effect on the promise score of the participant, which is determined and generated regarding the promise chain and displayed via the screen, wherein a level of sureness.

2. The promises-tracking device, as recited in claim 1, wherein the at least one memory module further instructs the at least one processor to determine whether the one of the plurality of viable promises being broken is the excused broken promise or the unexcused broken promise by the at least one processor, wherein only the unexcused broken promise has the negative effect on the promise score.

3. The promises-tracking device, as recited in claim 1, further comprising a selector which is configured to enable the participant to have a selection of a learning and training phase with one or more lessons for techniques of effective promise stored in the memory module according to a personal control score of the participant, wherein a baseline documentation is stored in the memory module and displayed via the screen, wherein the baseline documentation includes a plurality of personal, health and habitual questions for the participant to answer in order to obtain a personal control score for evaluating what kind of lesson and assignment in the learning and training phase to be displayed to the participant, wherein lesson material and suggestions are selected to the participant according to the personal control score through the selector, so as to help the participant to restore the sureness of the participant when said level of sureness drops.

4. The promises-tracking device, as recited in claim 2, further comprising a selector which is configured to enable the participant to have a selection of a learning and training phase with one or more lessons for techniques of effective promise stored in the memory module according to a personal control score of the participant, wherein a baseline documentation is stored in the memory module and displayed via the screen, wherein the baseline documentation includes a plurality of personal, health and habitual questions for the participant to answer in order to obtain a personal control score for evaluating what kind of lesson and assignment in the learning and training phase to be displayed to the participant, wherein lesson material and suggestions are selected to the participant according to the personal control score through the selector, so as to help the participant to restore the sureness of the participant when said level of sureness drops.

5. The promises-tracking device, as recited in claim 1, wherein the promise score contains information selected from the group consisting of total promises added to chain, total broken promise, and percentage of successful promise.

6. The promises-tracking device, as recited in claim 2, wherein the promise score contains information selected from the group consisting of total promises added to chain, total broken promise, and percentage of successful promise.

7. The promises-tracking device, as recited in claim 2, wherein the promise score contains information selected from the group consisting of total promises added to chain, total broken promise, and percentage of successful promise.

8. The promises-tracking device, as recited in claim 3, wherein the promise score contains information selected from the group consisting of total promises added to chain, total broken promise, and percentage of successful promise.

9. The promises-tracking device as recited in claim 4, wherein the promise score contains information selected from the group consisting of total promises added to chain, total broken promise, and percentage of successful promise.

10. The promises-tracking device, as recited in claim 1, wherein the promises-tracking device is a non-transitory computer readable medium containing computer instructions stores in the at least one memory module for causing the at least one processor to perform steps of:
recording the plurality of viable promises made by the participant in a chainlike manner through the input device to form the promise chain and storing the plurality of viable promises of the promise chain on the at least one memory module;
generating the physical activation signal through the activator to notify while the viable promises is sealed in the promise chain;
in response to setting promise times of the viable promises by the participant via the time setter, numbering the plurality of viable promises in an order in the memory module;
selectively notifying the participant through the screen when the promise times are up;
determining and generating the promise score of the participant regarding the promise chain, which is an incentive to motivate the participant to keep the promise score as high as possible, wherein when one of the viable promises is broken, the promise chain is then broken to have a negative effect on the promise score;
determining whether one of the plurality of viable promises being broken is an excused broken promise or an unexcused broken promise through the selectively input of one of the excused broken promise button and the unexcused broken promise button, wherein only the unexcused broken promise has the negative effect on the promise score; and
providing the level of sureness of the participant on the screen, wherein the level of sureness of the participant is indicated graphically on the screen for the participant to view and displayed periodically on the screen for monitoring the level of sureness of the participant.

11. A promises-tracking method for forming a promise chain and a promise score for tracking a plurality of viable promises from a participant, executed by a program operated electronic device which comprises a screen, an activator, at least one memory module, and an input device including a time setter, an excused broken promise button and an unexcused broken promise button, wherein the promises-tracking method comprises steps of:
(a) recording the plurality of viable promises made by the participant in a chainlike manner through the input device of the program operated electronic device to form the promise chain and storing the plurality of viable promises of the promise chain on the memory module of the program operated electronic device;
(b) generating a physical activation signal through the activator of the program operated electronic device to notify while the viable promises is sealed in the promise chain;
(c) in response to setting promise times of the plurality of viable promises by the participant via the time setter of the input device of the program operated electronic device, numbering the viable promises in an order in the memory module;
(d) selectively notifying the participant via the program operated electronic device when the promise times are up;
(e) determining and generating a promise score of the participant regarding the promise chain, which is an incentive to motivate the participant to keep the promise score as high as possible, wherein when one of the viable promises is broken, the promise chain is then broken;
(f) determining whether one of the plurality of viable promises being broken is an excused broken promise or an unexcused broken promise when one of the excused broken promise button and the unexcused broken promise button is selected to input by the participant, wherein only the unexcused broken promise is broken has a negative effect on the promise score; and
(g) providing a level of sureness of the participant on the screen, wherein the level of sureness of the participant is indicated graphically on the screen of the program operated electronic device for the participant to view and displayed periodically on the screen of the program operated electronic device for monitoring the level of sureness of the participant.

12. The promises-tracking method, as recited in claim 11, wherein the step (e) further comprises steps of:
(e1) receiving an input of a broken promise selectively by the participant via one of the excused broken promise button and the unexcused broken promise button of the input device of the program operated electronic device, and
(e2) displaying the promise score on the screen of the program operated electronic device.

13. The promises-tracking method, as recited in claim 11, wherein the step (g) further comprises a step of monitoring the level of sureness of the participant for ensuring the participant is able to keep the viable promises, wherein when the level of sureness drops, lesson material and suggestions are provided to help restore the sureness of the participant.

14. The promises-tracking method, as recited in claim 12, wherein the step (g) further comprises a step of monitoring the level of sureness of the participant for ensuring the participant is able to keep the viable promises, wherein when the level of sureness drops, lesson material and suggestions are provided to help restore the sureness of the participant.

15. The promises-tracking method, as recited in claim 11, wherein in the step (e), the promise score contains information selected from the group consisting of total promises added the promise chain, total broken promise and a percentage of successful promise.

16. The promises-tracking method, as recited in claim 12, wherein in the step (e), the promise score contains information selected from the group consisting of total promises added the promise chain, total broken promise and a percentage of successful promise.

17. The promises-tracking method, as recited in claim 14, wherein in the step (e), the promise score contains information selected from the group consisting of total promises added the promise chain, total broken promise and a percentage of successful promise.

18. The promises-tracking method, as recited in claim 11, further comprising a step of providing a learning and training phase by the program operated electronic device to the participant, wherein in the learning and training phase, one or more lessons are given to the participant regarding effective promise techniques, and one or more assignments for the participant to complete regarding the promise techniques, wherein an assignment score of the participant is determined regarding a completeness of the assignment.

19. The promises-tracking method, as recited in claim 11, wherein in responsive to a selection of the participant through a selector provided on the input device of the program operated electronic device, the program operated electronic device provides at least a learning and training phase with one or more lessons for techniques of effective promise stored in the memory module to the participant according to a personal control score of the participant.

20. The promises-tracking method, as recited in claim 19, wherein a baseline documentation phase is provided and stored in the memory module, wherein a plurality of personal, health and habitual questions are given for the participant to answer in order to obtain a personal control score for evaluating what kind of lesson and assignment in a learning and training phase to be given to the participant, wherein lesson material and suggestions are selected to the participant according to the personal control score through the selector, wherein the lesson material and suggestions are provided to the participant via the screen of the program operated electronic device to help the participant to restore the sureness of the participant when the level of sureness drops.
